SEQUENCE LISTING (18 sequences)

SEQ ID NO: 1 - protein, 399 aa, Homo sapiens
  REGION 1..399: note = misc_feature - human PDSS2 Sequence; >NP_065114.3 PDSS2 [organism=Homo sapiens] [GeneID=57107]
MNFRQLLLHLPRYLGASGSPRRLWWSPSLDTISSVGSWRGRSSKSPAHWNQVVSEAEKIVGYPTSFMSLRCLLSDELSNIAMQVRKLVGTQHPLLTTARGLVHDSWNSLQLRGLVVLLISKAAGPSSVNTSCQNYDMVSGIYSCQRSLAEITELIHIALLVHRGIVNLNELQSSDGPLKDMQFGNKIAILSGDFLLANACNGLALLQNTKVVELLASALMDLVQGVYHENSTSKESYITDDIGISTWKEQTFLSHGALLAKSCQAAMELAKHDAEVQNMAFQYGKHMAMSHKINSDVQPFIKEKTSDSMTFNLNSAPVVLHQEFLGRDLWIKQIGEAQEKGRLDYAKLRERIKAGKGVTSAIDLCRYHGNKALEALESFPPSEARSALENIVFAVTRFS

SEQ ID NO: 2 - protein, 465 aa, Homo sapiens
  REGION 1..465: note = misc_feature - human PDSS2 Sequence; >XP_011534258.1 PDSS2 [organism=Homo sapiens] [GeneID=57107] [isoform=X1]
MNFRQLLLHLPRYLGASGSPRRLWWSPSLDTISSVGSWRGRSSKSPAHWNQVVSEAEKIVGYPTSFMSLRCLLSDELSNIAMQVRKLVGTQHPLLTTARGLVHDSWNSLQLRGLVVLLISKAAGPSSVNTSCQNYDMVSGIYSCQRSLAEITELIHIALLVHRGIVNLNELQSSDGPLKDMQFGNKIAILSGDFLLANACNGLALLQNTKVVELLASALMDLVQGVYHENSTSKESYITDDIGISTWKEQTFLSHGALLAKSCQAAMELAKHDAEVQNMAFQYGKHMAMSHKINSDVQPFIKEKTSDSMTFNLNSAPVVLHQEFLGRDLWIKQIGEAQEKGRLDYAKERGLAVTQTGDAFFMTQRMPLGFLITEALDNGRDFHWKMAKRKIWCTNKSTPGPSNGSATNSMQNLLRERIKAGKGVTSAIDLCRYHGNKALEALESFPPSEARSALENIVFAVTRFS

SEQ ID NO: 3 - protein, 421 aa, Homo sapiens
  REGION 1..421: note = misc_feature - human PDSS2 Sequence; >XP_011534259.1 PDSS2 [organism=Homo sapiens] [GeneID=57107] [isoform=X2]
MNFRQLLLHLPRYLGASGSPRRLWWSPSLDTISSVGSWRGRSSKSPAHWNQVVSEAEKIVGYPTSFMSLRCLLSDELSNIAMQVRKLVGTQHPLLTTARGLVHDSWNSLQLRGLVVLLISKAAGPSSVNTSCQNYDMVSGIYSCQRSLAEITELIHIALLVHRGIVNLNELQSSDGPLKDMQFGNKIAILSGDFLLANACNGLALLQNTKVVELLASALMDLVQGVYHENSTSKESYITDDIGISTWKEQTFLSHGALLAKSCQAAMELAKHDAEVQNMAFQYGKHMAMSHKAQEKGRLDYAKERGLAVTQTGDAFFMTQRMPLGFLITEALDNGRDFHWKMAKRKIWCTNKSTPGPSNGSATNSMQNLLRERIKAGKGVTSAIDLCRYHGNKALEALESFPPSEARSALENIVFAVTRFS

SEQ ID NO: 4 - protein, 420 aa, Homo sapiens
  REGION 1..420: note = misc_feature - human PDSS2 Sequence; >XP_011534260.1 PDSS2 [organism=Homo sapiens] [GeneID=57107] [isoform=X3]
MNFRQLLLHLPRYLGASGSPRRLWWSPSLDTISSVGSWRGRSSKSPAHWNQVVSEAEKIVGYPTSFMSLRCLLSDELSNIAMQVRKLVGTQHPLLTTASQRSLAEITELIHIALLVHRGIVNLNELQSSDGPLKDMQFGNKIAILSGDFLLANACNGLALLQNTKVVELLASALMDLVQGVYHENSTSKESYITDDIGISTWKEQTFLSHGALLAKSCQAAMELAKHDAEVQNMAFQYGKHMAMSHKINSDVQPFIKEKTSDSMTFNLNSAPVVLHQEFLGRDLWIKQIGEAQEKGRLDYAKERGLAVTQTGDAFFMTQRMPLGFLITEALDNGRDFHWKMAKRKIWCTNKSTPGPSNGSATNSMQNLLRERIKAGKGVTSAIDLCRYHGNKALEALESFPPSEARSALENIVFAVTRFS

SEQ ID NO: 5 - protein, 355 aa, Homo sapiens
  REGION 1..355: note = misc_feature - human PDSS2 Sequence; >XP_011534261.1 PDSS2 [organism=Homo sapiens] [GeneID=57107] [isoform=X6]
MNFRQLLLHLPRYLGASGSPRRLWWSPSLDTISSVGSWRGRSSKSPAHWNQVVSEAEKIVGYPTSFMSLRCLLSDELSNIAMQVRKLVGTQHPLLTTARGLVHDSWNSLQLRGLVVLLISKAAGPSSVNTSCQNYDMVSGIYSCQRSLAEITELIHIALLVHRGIVNLNELQSSDGPLKDMQFGNKIAILSGDFLLANACNGLALLQNTKVVELLASALMDLVQGVYHENSTSKESYITDDIGISTWKEQTFLSHGALLAKSCQAAMELAKHDAEVQNMAFQYGKHMAMSHKAQEKGRLDYAKLRERIKAGKGVTSAIDLCRYHGNKALEALESFPPSEARSALENIVFAVTRFS

SEQ ID NO: 6 - protein, 329 aa, Homo sapiens
  REGION 1..329: note = misc_feature - human PDSS2 Sequence; >XP_011534262.1 PDSS2 [organism=Homo sapiens] [GeneID=57107] [isoform=X8]
MVSGIYSCQRSLAEITELIHIALLVHRGIVNLNELQSSDGPLKDMQFGNKIAILSGDFLLANACNGLALLQNTKVVELLASALMDLVQGVYHENSTSKESYITDDIGISTWKEQTFLSHGALLAKSCQAAMELAKHDAEVQNMAFQYGKHMAMSHKINSDVQPFIKEKTSDSMTFNLNSAPVVLHQEFLGRDLWIKQIGEAQEKGRLDYAKERGLAVTQTGDAFFMTQRMPLGFLITEALDNGRDFHWKMAKRKIWCTNKSTPGPSNGSATNSMQNLLRERIKAGKGVTSAIDLCRYHGNKALEALESFPPSEARSALENIVFAVTRFS

SEQ ID NO: 7 - protein, 297 aa, Homo sapiens
  REGION 1..297: note = misc_feature - human PDSS2 Sequence; >XP_011534263.1 PDSS2 [organism=Homo sapiens] [GeneID=57107] [isoform=X11]
MNFRQLLLHLPRYLGASGSPRRLWWSPSLDTISSVGSWRGRSSKSPAHWNQVVSEAEKIVGYPTSFMSLRCLLSDELSNIAMQVRKLVGTQHPLLTTARGLVHDSWNSLQLRGLVVLLISKAAGPSSVNTSCQNYDMVSGIYSCQRSLAEITELIHIALLVHRGIVNLNELQSSDGPLKDMQFGNKIAILSGDFLLANACNGLALLQNTKVVELLASALMDLVQGVYHENSTSKAQEKGRLDYAKLRERIKAGKGVTSAIDLCRYHGNKALEALESFPPSEARSALENIVFAVTRFS

SEQ ID NO: 8 - protein, 240 aa, Homo sapiens
  REGION 1..240: note = misc_feature - human PDSS2 Sequence; >XP_011534265.1 PDSS2 [organism=Homo sapiens] [GeneID=57107] [isoform=X14]
MNFRQLLLHLPRYLGASGSPRRLWWSPSLDTISSVGSWRGRSSKSPAHWNQVVSEAEKIVGYPTSFMSLRCLLSDELSNIAMQVRKLVGTQHPLLTTARGLVHDSWNSLQLRGLVVLLISKAAGPSSVNTSCQNYDMVSGIYSCQRSLAEITELIHIALLVHRGIVNLNELQSSDGPLKDMQFGNKIAILSGDFLLANACNGLALLQNTKSFSFNGPIAIYQMGDCESAWILSKHPRALS

SEQ ID NO: 9 - protein, 359 aa, Homo sapiens
  REGION 1..359: note = misc_feature - human PDSS2 Sequence; >XP_016866571.1 PDSS2 [organism=Homo sapiens] [GeneID=57107] [isoform=X5]
MNFRQLLLHLPRYLGASGSPRRLWWSPSLDTISSVGSWRGRSSKSPAHWNQVVSEAEKIVGYPTSFMSLRCLLSDELSNIAMQVRKLVGTQHPLLTTARGLVHDSWNSLQLRGLVVLLISKAAGPSSVNTSCQNYDMVSGIYSCQRSLAEITELIHIALLVHRGIVNLNELQSSDGPLKDMQFGNKIAILSGDFLLANACNGLALLQNTKVVELLASALMDLVQGVYHENSTSKESYITDDIGISTWKEQTFLSHGALLAKSCQAAMELAKHDAEVQNMAFQYGKHMAMSHKINSDVQPFIKEKTSDSMTFNLNSAPVVLHQEFLGRDLWIKQIGESSSTEVQLSNFQQMLLSTLLSTA

SEQ ID NO: 10 - protein, 363 aa, Homo sapiens
  REGION 1..363: note = misc_feature - human PDSS2 Sequence; >XP_047275052.1 PDSS2 [organism=Homo sapiens] [GeneID=57107] [isoform=X4]
MNFRQLLLHLPRYLGASGSPRRLWWSPSLDTISSVGSWRGRSSKSPAHWNQVVSEAEKIVGYPTSFMSLRCLLSDELSNIAMQVRKLVGTQHPLLTTARGLVHDSWNSLQLRGLVVLLISKAAGPSSVNTSCQNYDMVSGIYSCQRSLAEITELIHIALLVHRGIVNLNELQSSDGPLKDMQFGNKIAILSGDFLLANACNGLALLQNTKVVELLASALMDLVQGVYHENSTSKAQEKGRLDYAKERGLAVTQTGDAFFMTQRMPLGFLITEALDNGRDFHWKMAKRKIWCTNKSTPGPSNGSATNSMQNLLRERIKAGKGVTSAIDLCRYHGNKALEALESFPPSEARSALENIVFAVTRFS

SEQ ID NO: 11 - protein, 354 aa, Homo sapiens
  REGION 1..354: note = misc_feature - human PDSS2 Sequence; >XP_047275053.1 PDSS2 [organism=Homo sapiens] [GeneID=57107] [isoform=X7]
MNFRQLLLHLPRYLGASGSPRRLWWSPSLDTISSVGSWRGRSSKSPAHWNQVVSEAEKIVGYPTSFMSLRCLLSDELSNIAMQVRKLVGTQHPLLTTASQRSLAEITELIHIALLVHRGIVNLNELQSSDGPLKDMQFGNKIAILSGDFLLANACNGLALLQNTKVVELLASALMDLVQGVYHENSTSKESYITDDIGISTWKEQTFLSHGALLAKSCQAAMELAKHDAEVQNMAFQYGKHMAMSHKINSDVQPFIKEKTSDSMTFNLNSAPVVLHQEFLGRDLWIKQIGEAQEKGRLDYAKLRERIKAGKGVTSAIDLCRYHGNKALEALESFPPSEARSALENIVFAVTRFS

SEQ ID NO: 12 - protein, 313 aa, Homo sapiens
  REGION 1..313: note = misc_feature - human PDSS2 Sequence; >XP_047275054.1 PDSS2 [organism=Homo sapiens] [GeneID=57107] [isoform=X9]
MRDSGLVQAGWPRGLVHDSWNSLQLRGLVVLLISKAAGPSSVNTSCQNYDMVSGIYSCQRSLAEITELIHIALLVHRGIVNLNELQSSDGPLKDMQFGNKIAILSGDFLLANACNGLALLQNTKVVELLASALMDLVQGVYHENSTSKESYITDDIGISTWKEQTFLSHGALLAKSCQAAMELAKHDAEVQNMAFQYGKHMAMSHKINSDVQPFIKEKTSDSMTFNLNSAPVVLHQEFLGRDLWIKQIGEAQEKGRLDYAKLRERIKAGKGVTSAIDLCRYHGNKALEALESFPPSEARSALENIVFAVTRFS

SEQ ID NO: 13 - protein, 310 aa, Homo sapiens
  REGION 1..310: note = misc_feature - human PDSS2 Sequence; >XP_047275055.1 PDSS2 [organism=Homo sapiens] [GeneID=57107] [isoform=X10]
MNFRQLLLHLPRYLGASGSPRRLWWSPSLDTISSVGSWRGRSSKSPAHWNQVVSEAEKIVGYPTSFMSLRCLLSDELSNIAMQVRKLVGTQHPLLTTASQRSLAEITELIHIALLVHRGIVNLNELQSSDGPLKDMQFGNKIAILSGDFLLANACNGLALLQNTKVVELLASALMDLVQGVYHENSTSKESYITDDIGISTWKEQTFLSHGALLAKSCQAAMELAKHDAEVQNMAFQYGKHMAMSHKAQEKGRLDYAKLRERIKAGKGVTSAIDLCRYHGNKALEALESFPPSEARSALENIVFAVTRFS

SEQ ID NO: 14 - protein, 285 aa, Homo sapiens
  REGION 1..285: note = misc_feature - human PDSS2 Sequence; >XP_047275056.1 PDSS2 [organism=Homo sapiens] [GeneID=57107] [isoform=X12]
MQFGNKIAILSGDFLLANACNGLALLQNTKVVELLASALMDLVQGVYHENSTSKESYITDDIGISTWKEQTFLSHGALLAKSCQAAMELAKHDAEVQNMAFQYGKHMAMSHKINSDVQPFIKEKTSDSMTFNLNSAPVVLHQEFLGRDLWIKQIGEAQEKGRLDYAKERGLAVTQTGDAFFMTQRMPLGFLITEALDNGRDFHWKMAKRKIWCTNKSTPGPSNGSATNSMQNLLRERIKAGKGVTSAIDLCRYHGNKALEALESFPPSEARSALENIVFAVTRFS

SEQ ID NO: 15 - protein, 252 aa, Homo sapiens
  REGION 1..252: note = misc_feature - human PDSS2 Sequence; >XP_047275057.1 PDSS2 [organism=Homo sapiens] [GeneID=57107] [isoform=X13]
MNFRQLLLHLPRYLGASGSPRRLWWSPSLDTISSVGSWRGRSSKSPAHWNQVVSEAEKIVGYPTSFMSLRCLLSDELSNIAMQVRKLVGTQHPLLTTASQRSLAEITELIHIALLVHRGIVNLNELQSSDGPLKDMQFGNKIAILSGDFLLANACNGLALLQNTKVVELLASALMDLVQGVYHENSTSKAQEKGRLDYAKLRERIKAGKGVTSAIDLCRYHGNKALEALESFPPSEARSALENIVFAVTRFS

SEQ ID NO: 16 - DNA, 1206 bp, Mus musculus
  misc_feature 1..1206: note = Mouse Nucleotide Sequence
  source 1..1206: mol_type = genomic DNA
atgagcctccggcagctgctgttgcgcttgtccggttacctcggggcttcaggtccccccagtcgccactggtggtacttcagatccctcgacagcatctcctcggcgggctcctggcgcgggcgctcctccaggtcaccggcccattggaaccaggtggtgtccgaggcggagaagatcgtgggctaccccgcatccttcatgagcctgcgctgcctgctgagcgacgagctcagcaatatcgccatgcaggtgcggaagctggtggggacgggacaccctctgcttaccactgccagggccctcgtgcacgacagccggcataacctacaactgcggggcctggtcgtgctcctcatatcaaaggctgcggggcccagcactcggaacgctgcgtgtcagaactacgacatggtcagtggggtatactcatgtcaaagaagtttggcagagatcacagaacttatccatactgctctcctggtgcatcgtgggatagtaaacttaagtgaattacagtcatctgatggaccactgaaagacatgcagtttggaaacaaaatagctatcctgagtggagacttccttctagcaaatgcatgcaatggactagctcttctacagaacaccaaggttgtggagcttttatcaagtgctcttatggacttggtgcatggagtataccaggagaactctgcttccaccaaggaaaattctatcccagatgatattggaatctcgacctggaaggagcagactttcctgtcccattgtgccttgctagcgaagagctgccaggctgcaatggagttagcaaagcatgatgctgcggtccaagacatggcattccagtatgggaagcacatggccatgagtcacaagatcaatgctgacctccagccttttattaaagacaaggccagtgactctaagacttttaacctaaactcagcacctgtagtcttacatcaggagtttcttggaagagatttgtggattaagcagattggagaggctcaagagaaaggaagcttgaactacagtaagttgcgagaaacaatcaaagctggcaaaggtgtgacttcagctattgacctgtgtcgttaccatggaaacaaggcactagaggccctggagagcttccctccctcagaggccagatcggctttagaaaacattgtgtttgctgtgaccagattttcttga

SEQ ID NO: 17 - protein, 401 aa, Mus musculus
  REGION 1..401: note = misc_feature - Mouse Translation
MSLRQLLLRLSGYLGASGPPSRHWWYFRSLDSISSAGSWRGRSSRSPAHWNQVVSEAEKIVGYPASFMSLRCLLSDELSNIAMQVRKLVGTGHPLLTTARALVHDSRHNLQLRGLVVLLISKAAGPSTRNAACQNYDMVSGVYSCQRSLAEITELIHTALLVHRGIVNLSELQSSDGPLKDMQFGNKIAILSGDFLLANACNGLALLQNTKVVELLSSALMDLVHGVYQENSASTKENSIPDDIGISTWKEQTFLSHCALLAKSCQAAMELAKHDAAVQDMAFQYGKHMAMSHKINADLQPFIKDKASDSKTFNLNSAPVVLHQEFLGRDLWIKQIGEAQEKGSLNYSKLRETIKAGKGVTSAIDLCRYHGNKALEALESFPPSEARSALENIVFAVTRFS

SEQ ID NO: 18 - DNA, 1773 bp, Mus musculus
  misc_feature 1..1773: note = Mouse PDSS2 RNA
  source 1..1773: mol_type = genomic DNA
atgcctccaacatggcgccccccatgtaggcactggctgtggttaccagcgcaggtatccagtgcagttctgagcagccgctggggttacgccagtctgcgctgcagaagcagcaaacgttattcctggttacggctccagaaagccatacttctctgagggtttggaatcgccgtctcctgttagattctgaaagtttctcctcagcccttgtttgcttcccgtgtcatttctggccaccttcctcaggtccccgaccctctcagactcaaaactatgagcctccggcagctgctgttgcgcttgtccggttacctcggggcttcaggtccccccagtcgccactggtggtacttcagatccctcgacagcatctcctcggcgggctcctggcgcgggcgctcctccaggtcaccggcccattggaaccaggtggtgtccgaggcggagaagatcgtgggctaccccgcatccttcatgagcctgcgctgcctgctgagcgacgagctcagcaatatcgccatgcaggtgcggaagctggtggggacgggacaccctctgcttaccactgccagggccctcgtgcacgacagccggcataacctacaactgcggggcctggtcgtgctcctcatatcaaaggctgcggggcccagcactcggaacgctgcgtgtcagaactacgacatggtcagtggggtatactcatgtcaaagaagtttggcagagatcacagaacttatccatactgctctcctggtgcatcgtgggatagtaaacttaagtgaattacagtcatctgatggaccactgaaagacatgcagtttggaaacaaaatagctatcctgagtggagacttccttctagcaaatgcatgcaatggactagctcttctacagaacaccaaggttgtggagcttttatcaagtgctcttatggacttggtgcatggagtataccaggagaactctgcttccaccaaggaaaattctatcccagatgatattggaatctcgacctggaaggagcagactttcctgtcccattgtgccttgctagcgaagagctgccaggctgcaatggagttagcaaagcatgatgctgcggtccaagacatggcattccagtatgggaagcacatggccatgagtcacaagatcaatgctgacctccagccttttattaaagacaaggccagtgactctaagacttttaacctaaactcagcacctgtagtcttacatcaggagtttcttggaagagatttgtggattaagcagattggagaggctcaagagaaaggaagcttgaactacagtaagttgcgagaaacaatcaaagctggcaaaggtgtgacttcagctattgacctgtgtcgttaccatggaaacaaggcactagaggccctggagagcttccctccctcagaggccagatcggctttagaaaacattgtgtttgctgtgaccagattttcttgacaccaaattaaaaagacactattgttccttagctgaaaaacccagagaatgaggcggacaggagagcttttggtgatgaaataagtaacgtgtttaatgactttagaaatatatgtattttttttttctgtcctttatctgactgcttaatgaatctatcctttggaaccactacaaacaaaacgagaaggaaaaaggtcacgcggttttcacttgtcacgccagagcacacttgaggcttcagcaacagaaataattaatgagactcgctcccgtgacctcagcaaatgg

(19) United States
(12) Patent Application Publication
(10) Pub. No.: US 2026/0240995 A1
(43) Pub. Date: Aug. 20, 2026

(54) ENHANCING CAR-T CELL PERFORMANCE FOR CANCER IMMUNOTHERAPY

(71) Applicant: THE REGENTS OF THE UNIVERSITY OF CALIFORNIA, Oakland, CA (US)

(72) Inventors: Ananda GOLDRATH, La Jolla, CA (US); Miguel REINA-CAMPOS, La Jolla, CA (US)

(21) Appl. No.: 19/161,641

(22) PCT Filed: Feb. 29, 2024
(86) PCT No.: PCT/US2024/017796

§ 371 Date: Sep. 2, 2025

Related U.S. Application Data
(60) Provisional application No. 63/449,701, filed on Mar. 3, 2023.

(51) Int. Cl. A61K 40/11; A61K 38/00; A61K 40/31; A61K 40/42; C07K 14/705; C07K 16/00; C12N 5/0783; C12N 9/10

(52) U.S. Cl. CPC A61K 40/11; A61K 40/31; A61K 40/421; C07K 14/70517; C07K 16/00; C12N 5/0636; C12N 9/1085; C12Y 205/01091; A61K 38/00; C12N 2510/00

(57) ABSTRACT

Provided are methods for metabolically enhancing chimeric antigen receptor (CAR)-T cell function and persistence, wherein the enhancing of CAR-T cells can be used for the enhancement of CAR-T cell efficacy in cancer immunotherapy, and provided are products of manufacture and kits comprising CAR-T cells genetically altered to have greater than wild type or substantially more expression or activity of decaprenyl-diphosphate synthase subunit 2 (PDSS2). Provided are methods for metabolically enhancing chimeric antigen receptor (CAR)-T cell function and persistence comprising administering a CAR-T cell with a heterologous gene expressing PDSS2. Provided herein are CAR-T cells that have been treated in vivo or ex vivo with a nucleic acid to increase the activity of a PDSS2 in the CAR-T cell. Provided are methods for metabolically enhancing CD8 T cells resulting in their having improved metabolic fitness and increased accumulation in vivo in response to infection and tumors.

### RELATED APPLICATIONS

This Patent Convention Treaty (PCT) International Application claims the benefit of priority under 35 U.S.C. § 119(e) of U.S. Provisional Patent Application Ser. No. (USSN) 63/449,701, Mar. 3, 2023. The aforementioned patent application is expressly incorporated herein by reference in its entirety and for all purposes. All publications, patents, patent applications cited herein are hereby expressly incorporated by reference for all purposes.

### STATEMENT AS TO FEDERALLY SPONSORED RESEARCH

This invention was made with government support under R01AI067545 awarded by the National Institutes of Health (NIH). The government has certain rights in the invention.

### REFERENCE TO ELECTRONIC SEQUENCE LISTING

The application contains a Sequence Listing which has been submitted electronically in XML format and is hereby incorporated by reference in its entirety. Said .XML copy, created on Feb. 28, 2024, is named “0321.151668PCT.xml” and is 33,052 bytes in size. The sequence listing contained in this .XML file is part of the specification and is hereby incorporated by reference herein in its entirety.

### TECHNICAL FIELD

This invention generally relates to cell biology and cancer therapies. In alternative embodiments, provided are methods for metabolically enhancing chimeric antigen receptor (CAR)-T cell function and persistence, wherein the enhancing of CAR-T cells can be used for the enhancement of CAR-T cell efficacy in cancer immunotherapy. In alternative embodiments, provided are products of manufacture and kits comprising CAR-T cells genetically altered to have greater than wild type or substantially more expression or activity of decaprenyl-diphosphate synthase subunit 2 (PDSS2), an enzyme that synthesizes the prenyl side-chain of coenzyme Q, or ubiquinone, one of the key elements in the respiratory chain. In alternative embodiments, provided are methods for metabolically enhancing chimeric antigen receptor (CAR)-T cell function and persistence comprising administering a CAR-T cell with a heterologous gene expressing PDSS2. In alternative embodiments, provided herein are CAR-T cells that have been treated in vivo or ex vivo (before being administered in vivo) with an agent, which for example can be a nucleic acid or a small molecule, to increase the activity of a PDSS2 in the CAR-T cell. In alternative embodiments, provided are methods for metabolically enhancing CD8 T cells resulting in their having improved metabolic fitness and increased accumulation in vivo in response to infection and tumors.

### BACKGROUND

Chimeric antigen receptor (CAR) (CARs are also known as chimeric immunoreceptors, chimeric T cell receptors or artificial T cell receptors) T cell (CAR-T cell) therapies can be very effective in treating cancers and other diseases by their ability to target a specific antigen, for example, a cancer antigen.

CARs are receptor proteins that have been engineered to give T cells (particularly T cells, a type of lymphocyte, that can kill a cancer cell when the T cell specifically binds to that cancer cell, such as for example, a CD8+ T cell) a new ability to target a specific antigen such as a cancer antigen (a molecule just found on a cancer cell). The CAR receptors are chimeric because they combine both antigen-binding and T cell activating (for example, cancer killing) functions into a single receptor. Thus, after CAR T cells are infused into a patient, they act as a “living drug” against cancer cells. When the CAR-T cells come in contact with their targeted antigen on a cancer cell, CAR T cells bind to the cancer cell and become activated, then proceed to proliferate and become cytotoxic to the cancer cell.

Decaprenyl-diphosphate synthase subunit 2 (PDSS2) is an enzyme that synthesizes the prenyl side-chain of coenzyme Q, or ubiquinone, one of the key elements in the respiratory chain. The gene product catalyzes the formation of all trans-polyprenyl pyrophosphates from isopentyl diphosphate in the assembly of polyisoprenoid side chains, the first step in coenzyme Q biosynthesis.

### SUMMARY

In alternative embodiments, provided are methods for metabolically enhancing chimeric antigen receptor (CAR)-T cell, including CD8 T cells which target cancer cells, function and persistence, wherein the enhancing of CAR-T cells can be used for the enhancement of CAR-T cell efficacy in cancer immunotherapy, or for metabolically enhancing CD8 T cells resulting in their having improved metabolic fitness and increased accumulation in vivo in response to infection and tumors.

In alternative embodiments, provided are CAR-T cells or a pharmaceutical formulation comprising a CAR-T cell, wherein the CAR-T cell is genetically modified to have increased expression of, or substantially more expression or activity of, or more than wild type levels of expression or activity of, a decaprenyl-diphosphate synthase subunit 2 (PDSS2); and optionally the CAR-T cell is formulated as a pharmaceutical, or formulated for in vivo administration.

Overexpression of Pdss2 will improve the metabolic fitness of mouse CD8 T cells and increase their accumulation in vivo in response to infection and tumors. Human CAR-T cells can be engineered to overexpress PDSS2 to improve their in vivo accumulation and enhance their antitumor function.

In alternative embodiments, provided are CAR-T cells or a pharmaceutical formulation comprising a CAR-T cell, wherein the CAR-T cell comprises or has contained therein a compound or composition capable of generating a phenotype of: increased expression of, or substantially more expression or activity of, or more than wild type levels of expression or activity of, a PDSS2 enzyme or gene; and optionally the CAR-T cell is formulated or manufactured as or with a pharmaceutical, an implant, a device or is formulated for in vivo administration.

In alternative embodiments, provided are products of manufacture or kit, or a pharmaceutical formulation, comprising a CAR-T cell as provided herein.

In alternative embodiments, provided are CD8 cells or CAR-T cells or a pharmaceutical formulation comprising a CD8 cell or a CAR-T cell, wherein the CD8 cell or a CAR-T cell is genetically modified to have increased expression of, or substantially increased expression or activity of, or more than wild type levels of expression or activity of, a decaprenyl-diphosphate synthase subunit 2 (PDSS2) enzyme or gene.

In alternative embodiments of CD8 cells or CAR-T cells or a pharmaceutical formulation as provided herein the CD8 cell or CAR-T cell is formulated as a pharmaceutical, or formulated for in vivo administration.

In alternative embodiments, provided are CD8 cells or CAR-T cells or a pharmaceutical formulation comprising a CAR-T cell, wherein the CAR-T cell comprises or has contained therein a compound or composition capable of generating a phenotype of: increased expression of, or substantially increased expression or activity of, or more than wild type levels of expression or activity of, a decaprenyl-diphosphate synthase subunit 2 (PDSS2) enzyme or gene.

In alternative embodiments of CD8 cells or CAR-T cells or a pharmaceutical formulation as provided herein:
the CD8 cell or CAR-T cell further comprises, or has implanted, a heterologous nucleic acid encoding a protein having decaprenyl-diphosphate synthase subunit 2 (PDSS2) enzyme function;
the PDSS2 enzyme or gene is a human enzyme or gene;
the PDSS2 enzyme has a sequence as set forth in any of SEQ ID NO: 1 to SEQ ID NO:15, or the heterologous nucleic acid encodes a protein having an amino acid sequence as set forth in any of SEQ ID NO: 1 to SEQ ID NO:15;
CD8 cell or CAR-T cell further comprise or have implanted or contained therein an expression vehicle or a vector having the heterologous nucleic acid contained therein, and the heterologous nucleic acid expresses a PDSS2 enzyme in the CD8 cell or CAR-T cell; and optionally the expression vehicle or the vector is or comprises an expression construct, a plasmid, an expression vehicle, or a virus; and optionally the expression vehicle, vector or virus is or is derived from: a herpes simplex virus, a human immunodeficiency virus (HIV), a synthetic vector, an adeno-associated virus (AAV), a lentivirus, an adenovirus or a plasmid, and optionally the AAV is or is derived from: an AAV1, AAV2, AAV3, AAV4, AAV5, AAV6, AAV7, AAV8, AAV9, AAV10 AAV11, AAV12, pseudotyped AAV, a rhesus-derived AAV, AAVrh8, AAVrh10 and AAV-DJan AAV capsid mutant, an AAV hybrid serotype, an organ-tropic AAV, a cardiotropic AAV, or a cardiotropic AAVM41 mutant;
the PDSS2 enzyme-expressing nucleic acid is an RNA or a DNA molecule, or a synthetic nucleic acid; and/or
the CAR-T cell is formulated or manufactured as or with a pharmaceutical, an implant, a device or is formulated for in vivo administration.

In alternative embodiments, provided are products of manufacture or kits, or a pharmaceutical formulation, comprising a CD8 cell or CAR-T cell as provided herein, and optionally the product of manufacture is an implant or a device.

In alternative embodiments provided are methods for metabolically enhancing a CD8 T cell or a CAR-T cell resulting in the CD8 T cell or the CAR-T having improved metabolic fitness, and metabolically enhancing chimeric antigen receptor (CAR)-T cell function and persistence in vitro or in vivo, the method comprising inserting in the CD8 T cell or the CAR-T cell (or transfecting the CD8 T cell or the CAR-T cell) a heterologous nucleic acid encoding a protein having decaprenyl-diphosphate synthase subunit 2 (PDSS2) enzyme function.

In alternative embodiments of methods as provided herein:
the PDSS2 enzyme has a sequence as set forth in any of SEQ ID NO: 1 to SEQ ID NO: 15, or the heterologous nucleic acid encodes a protein having an amino acid sequence as set forth in any of SEQ ID NO:1 to SEQ ID NO:15;
the heterologous nucleic acid encoding the protein having PDSS2 enzyme function is contained in an expression vehicle or a vector; and/or
the expression vehicle, vector or virus is or is derived from: a herpes simplex virus, a human immunodeficiency virus (HIV), a synthetic vector, an adeno-associated virus (AAV), a lentivirus, an adenovirus or a plasmid.

In alternative embodiments provided are methods for metabolically enhancing CD8 T cells resulting in their having improved metabolic fitness and increased accumulation in vivo in response to infection and tumors, metabolically enhancing chimeric antigen receptor (CAR)-T cell function and persistence in vitro or in vivo, or enhancing CAR-T cell efficacy in cancer immunotherapy, the method comprising:
(a) administering to an individual in need thereof a CD8 cell or a CAR-T cell having increased or enhanced expression of, or substantially more expression or activity of, or more than wild type levels of expression or activity of, a PDSS2 enzyme or gene,
wherein optionally the CD8 cell or CAR-T cell has increased expression of nucleic acids encoding PDSS2,
and optionally the CAR-T cell is contacted with nucleic acids encoding PDSS2 before in vivo administration to an individual in need thereof, during or with in vivo administration of the CD8 cell or CAR-T cell, and/or after in vivo administration of the CD8 cell or CAR-T cell; and/or
(b) administering to an individual in need thereof a CD8 cell or CAR-T cell that has been engineered to have increased or enhanced expression of, or substantially more expression or activity of, or more than wild type levels of expression or activity of, a PDSS2 enzyme or gene,
wherein optionally the CD8 cell or CAR-T cell has increased expression of nucleic acids encoding PDSS2; and/or
(c) (b) administering to an individual in need thereof a CD8 cell and/or a CAR-T cell that has been genetically modified to have a phenotype comprising: having increased or enhanced expression of, or substantially more expression or activity of, or more than wild type levels of expression or activity of, a PDSS2 enzyme or gene.

In alternative embodiments of methods as provided herein:
the CD8 cell or CAR-T cell is administered by intravenous injection, subcutaneous injection, intrathecal injection, intramuscular injection; and/or between about 10² and 10¹⁰ CD8 cells or CAR-T cells (or a CD8 cell and/or a CAR-T cell that has been genetically modified as provided herein) are administered to the individual in need thereof.

In alternative embodiments, provided are uses of a product of manufacture or kit, or a pharmaceutical formulation, comprising a CD8 cell or CAR-T cell as provided herein:
for metabolically enhancing CD8 T cells resulting in their having improved metabolic fitness and increased accumulation in vivo in response to infection and tumors,
metabolically enhancing chimeric antigen receptor (CAR)-T cell function and persistence in vitro or in vivo, or
enhancing CAR-T cell efficacy in cancer immunotherapy.

In alternative embodiments, provided are products of manufacture or kit, or a pharmaceutical formulation as provided herein, for use in:
for metabolically enhancing CD8 T cells resulting in their having improved metabolic fitness and increased accumulation in vivo in response to infection and tumors,
metabolically enhancing chimeric antigen receptor (CAR)-T cell function and persistence in vitro or in vivo, or
enhancing CAR-T cell efficacy in cancer immunotherapy.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIGS. 1A and 1B illustrate Actionable Metabolic Interventions to Enhance Antitumor CD8 T Cell Function.

FIG. 2 illustrates the mevalonate/cholesterol synthesis pathway and exemplary mechanisms explaining how overexpression of Pdss2 will improve the metabolic fitness of mouse CD8 T cells and increase their accumulation in vivo in response to infection and tumors, and that human CAR-T cells can be engineered to overexpress PDSS2 to improve their in vivo accumulation and enhance their antitumor function.

FIG. 3A-C illustrate data showing that enforced over expression of Pdss2 on mouse P14 CD8 T cells increases their ability to accumulate in vivo after LCMV Armstrong infection:

FIG. 3A schematically illustrates an exemplary experimental setting;

FIG. 3B graphically illustrates qPCR of Pdss2 transcript on transduced mouse P14 CD8 T cells; and

FIG. 3C graphically illustrates: Ratio of Pdss2 OE cells vs control (Empty) cells at day 7 after infection with LCMV Armstrong; IV=Intravascular staining with anti-CD8a; Ab denotes vascular association at the time of analysis; and One sample T-Test against a 0 value.

FIG. 4A-B shows that gain of mitochondrial function upon Fdft1 deletion depends on Pdss2 activity; and enforcing mouse Pdss2 expression by retroviral transduction on mouse CD8 T cells increases mitochondrial function (Mitochondrial Stress Test (Seahorse); O=Oligomycin, F=FCCP, R/A=Rotenone/Antimycin A):

FIG. 4A graphically illustrates: assessment of the mitochondrial function of mouse CD8 T cells modified as control (sgCd19) Fdft1 KO (sgFdft1) and Fdft1 and Pdss2 KO (sgFdft1/sgPdss2); and

FIG. 4B graphically illustrates: assessment of the mitochondrial function of mouse CD8 T cells modified as control (Empty), and Pdss2 overexpressing cells.

FIG. 5 graphically illustrates that Pdss2 is required for the increased accumulation and is endowed by the loss of Fdft1 in subcutaneous tumors in mice (MC38-GP33-41 model).

FIG. 6 graphically illustrates Pdss2 is required for the increased accumulation endowed by the loss of Zc3h12a (aka Regnase-1) in subcutaneous tumors in mice (B16 melanoma model).

FIG. 7 illustrates Mouse Pdss2 mRNA (SEQ ID NO:18) and primers used to clone mouse PDSS2 from mouse liver cDNA.

FIG. 8 graphically illustrates that overexpression of mouse PDSS2 in tumor antigen-specific CD8 T cells enhances their tumor control ability in a melanoma tumor model: adoptive transfer of P14 CD8 T cells modified with either Empty (control) or mouse Pdss2-overexpressing constructs (Pdss2 OE) into tumor-bearing mice at day 8 after tumor implantation.

FIG. 9A-B graphically illustrate that overexpression of mouse PDSS2 enhanced accumulation of tumor antigen-specific CD8 T cells in the tumor and promotes features of tissue-resident-like CD8 T cells; adoptive transfer of a 1:1 mix of P14 CD8 T cells modified with either Empty (control) or mouse Pdss2-overexpressing constructs (Pdss2 OE) into tumor-bearing mice at day 8 post tumor implantation. Analysis performed at day 5 after P14 CD8 T cell transfer. MC38-GP33-41 model;

FIG. 9A graphically illustrates frequency of congenics;

FIG. 9B graphically illustrates frequency of T_{RM}-like TL.

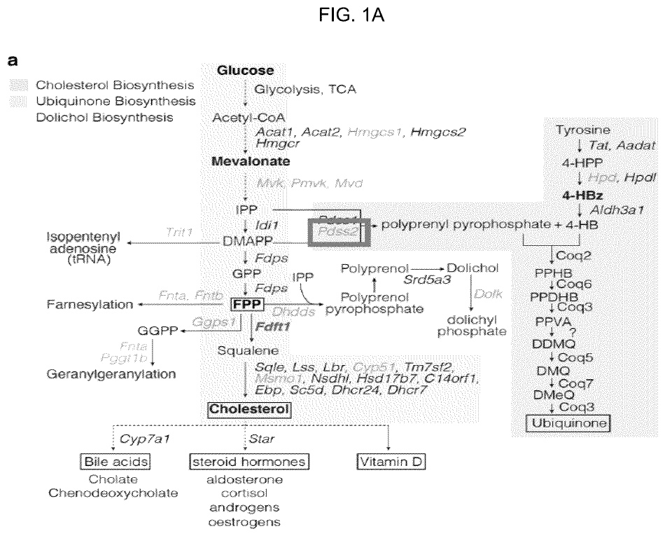

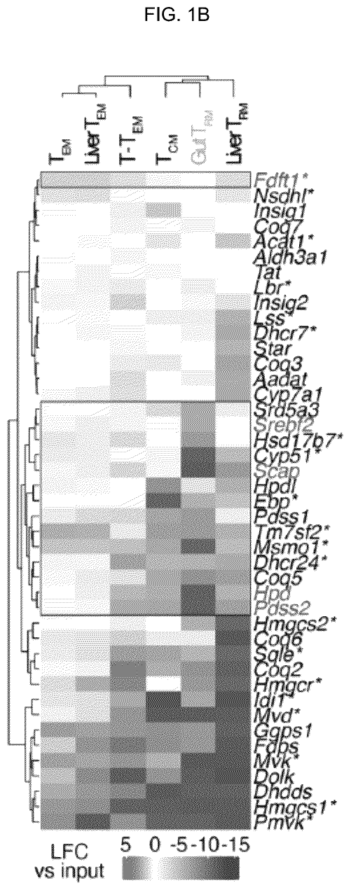

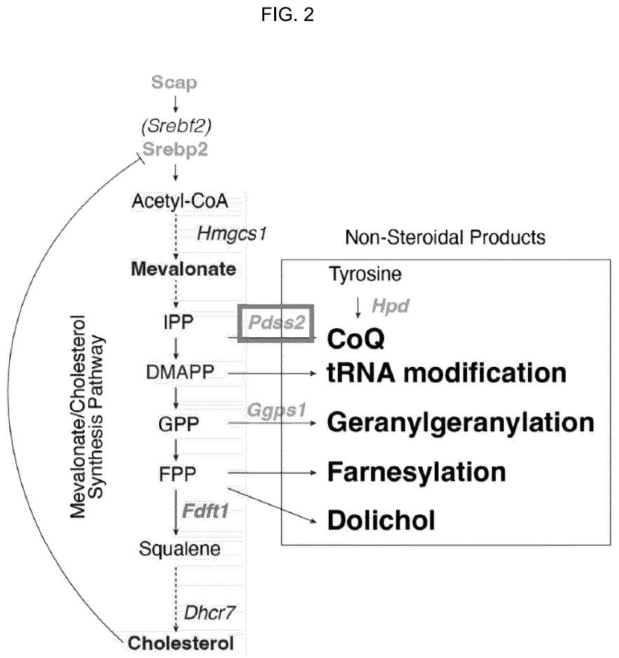

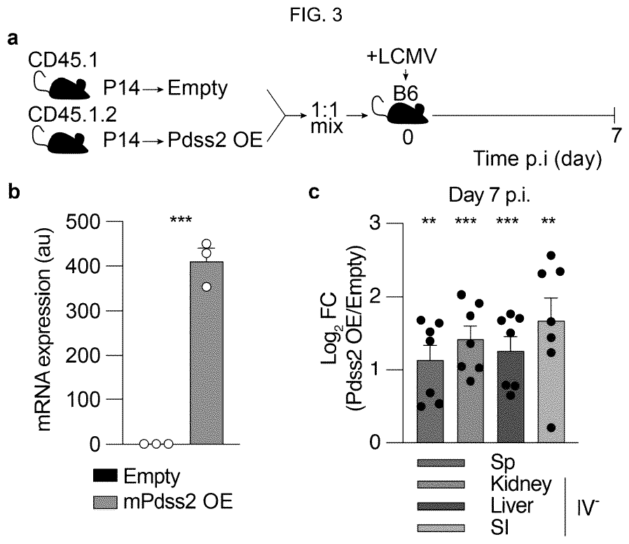

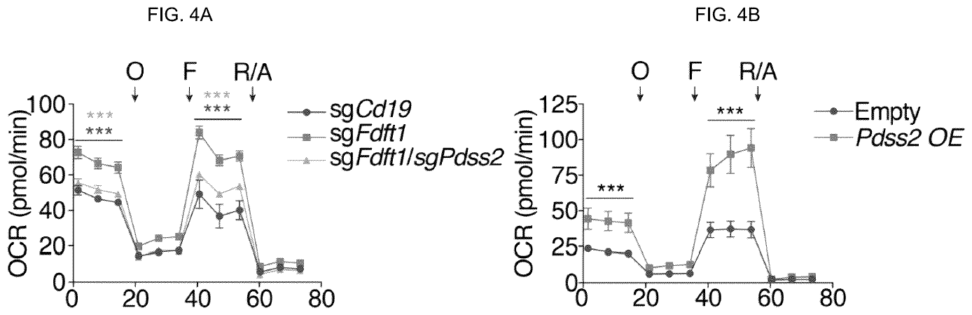

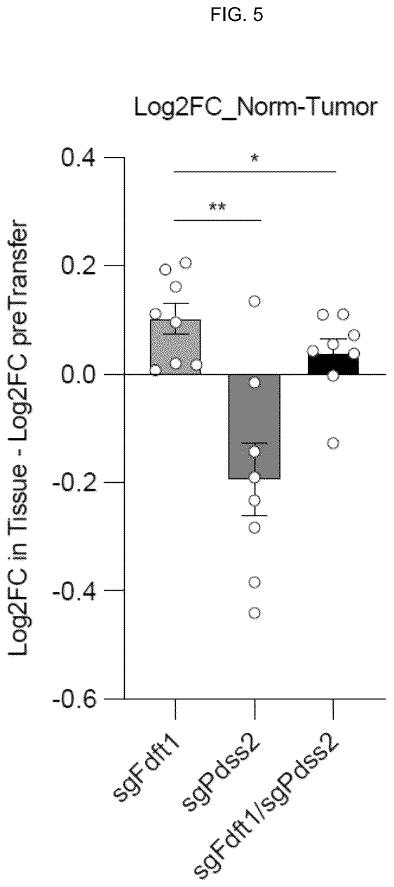

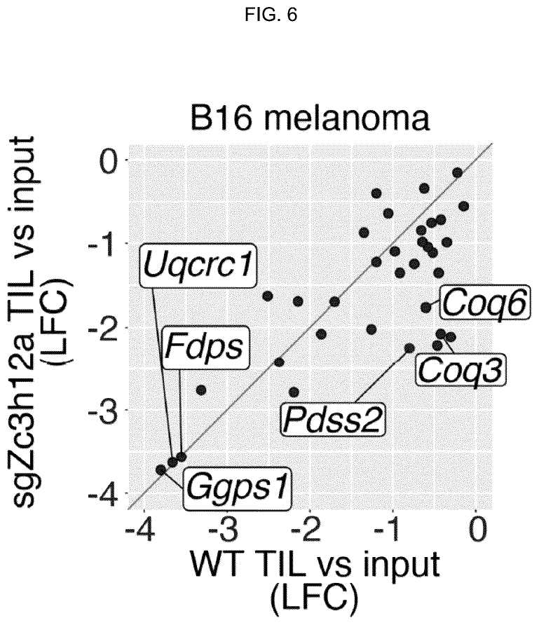

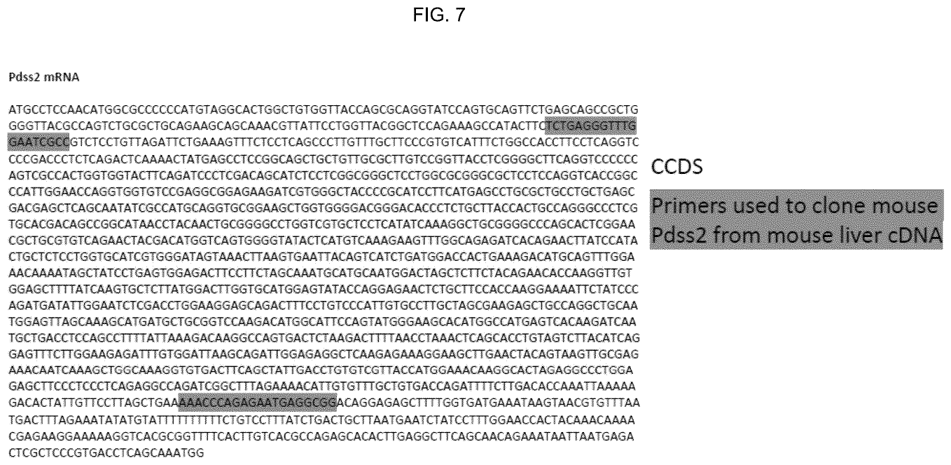

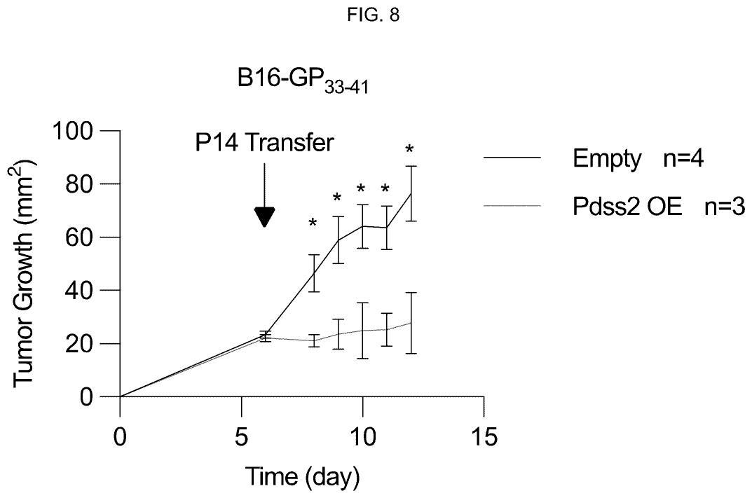

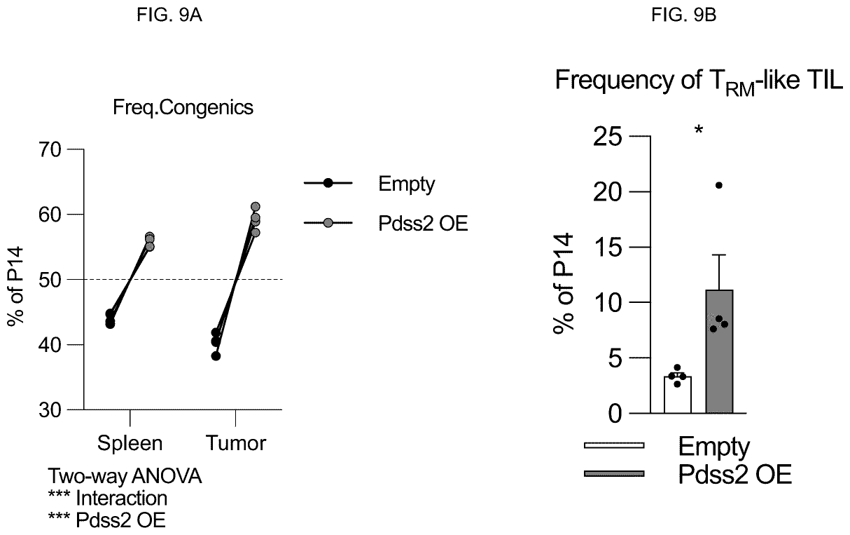

Like reference symbols in the various drawings indicate like elements.

### DETAILED DESCRIPTION

In alternative embodiments, provided are methods for metabolically enhancing CAR-T cell and CD8 T cell function in vitro and in vivo, and their persistence in vivo, wherein the enhancing of CD8 T cells and CAR-T cells can be used for the enhancement of CAR-T and CD8 T cell efficacy in cancer immunotherapy.

In alternative embodiments, provided are products of manufacture and kits comprising CD8 T cells CAR-T cells genetically altered to have greater than wild type or substantially more expression or activity of decaprenyl-diphosphate synthase subunit 2 (PDSS2). In alternative embodiments, provided are methods for metabolically enhancing chimeric antigen receptor (CAR)-T cell function and persistence comprising administering a CAR-T cell with a heterologous gene expressing PDSS2. In alternative embodiments, provided herein are CAR-T cells that have been treated in vivo or ex vivo (before being administered in vivo) with an agent, which for example can be a nucleic acid or a small molecule, to increase the activity of a PDSS2 in the CD8 T cells or CAR-T cell. In alternative embodiments, provided are products of manufacture and kits comprising CD8 T cells having improved metabolic fitness and increased accumulation in vivo in response to infection and tumors.

### CAR T Cell Production and Administration

CAR T-cells can be produced using any known protocol, for example, see for example U.S. Pat. Nos. 11,485,750; 11,472,858; 11,471,490; 11,471,519.

For example, the first step in the production of CAR T-cells is the isolation of T cells from human blood. CAR T-cells may be manufactured either from the patient's own blood, known as an autologous treatment, or from the blood of a healthy donor, known as an allogeneic treatment. The manufacturing process is the same in both cases; only the choice of initial blood donor is different.

Leukocytes can be isolated using a blood cell separator in a process known as leukocyte apheresis. Peripheral blood mononuclear cells (PBMCs) are then separated and collected. The products of leukocyte apheresis are then transferred to a cell-processing center, where specific T cells are stimulated so that they will actively proliferate and expand to large numbers. To drive their expansion, T cells can be typically treated ex vivo with the cytokine interleukin 2 (IL-2) and anti-CD3 antibodies. In alternative embodiments, the T cells are treated with a squalene synthase (or Fdft1) at this stage of the process.

Expanded T cells are then purified and then transduced with a gene encoding the engineered CAR via a retroviral vector, typically either an integrating gammaretrovirus (RV) or a lentiviral (LV) vector. These vectors are modified to be safe, for example, to have a partial deletion of their U3 region. Editing tools such as CRISPR/Cas9 can be used instead of retroviral vectors to integrate the CAR gene into specific sites in the genome. In alternative embodiments, the T cells are also genetically altered to have deleted or decreased ability to express squalene synthase (or Fdft1), or have diminished squalene synthase (or Fdft1) activity. The genetic sequences used to delete or modify squalene synthase (or Fdft1) gene can be on the same or different vectors or editing tools (for example, CRISPR/Cas9) as the engineered CAR.

The patient can be lymphodepleted, for example, by chemotherapy, prior to the introduction of the engineered CAR T-cells. The depletion of the number of circulating leukocytes in the patient can upregulate the number of cytokines that are produced and reduces competition for resources, which helps to promote the expansion of the engineered CAR T-cells. In alternative embodiments, before, in conjunction with, and/or after in vivo administration of the engineered CAR T-cells, inhibitors of squalene synthase (or Fdft1) nucleic acid and/or protein (enzyme) are also administered to the individual.

Any new or known CAR T cell can be used to practice methods as provided herein, for example, tisagenlecleucel (or KYMRIAH™, Novartis) axicabtagene ciloleucel (or YESCARTA™, Kite Pharma/Gilead), lisocabtagene maraleucel (or BREYANZI™, Juno Therapeutics/BMS), idecabtagene vicleucel (or ABECMA™, Bluebird Bio/BMS), and the like.

### Therapies

In alternative embodiments, CAR T cells used in practicing methods as provided herein are used to treat cancers or tumors, for example, where the CAR T cells can eradicate a certain cell population, for instance, a cancer such as a lymphoma, or any tumor, including solid tumors, or a non-solid tumor such as a blood cancer.

In alternative embodiments, CAR T cells used in practicing methods as provided herein are used to treat or ameliorate an autoimmune disease. For example, T cells can also mediate tolerance to antigens, and a regulatory T cell genetically modified to express a CAR could have the potential to confer tolerance to a specific antigen, a property that can be utilized in organ transplantation or rheumatic diseases like lupus.

In alternative embodiments, CD8 T cells and/or CAR T cells and provided herein, and CD8 T cells and/or CAR T cells used in practicing methods as provided herein are genetically modified to express more, substantially more, or more than wild type levels of PDSS2 enzyme.

In alternative embodiments, nucleic acids (optionally coding sequences such as transcripts, and/or genes) encoding any of the following human PDSS2 enzyme variants or isoforms are used to make CD8 T cells and/or CAR T cells as provided herein and to practice methods as provided herein:

(SEQ ID NO: 1) human PDSS2 Sequence:
>NP_065114.3 PDSS2 [organism = Homo sapiens] [GeneID = 57107]
MNFRQLLLHLPRYLGASGSPRRLWWSPSLDTISSVGSWRGRSSKSPAHWNQVVSEAEKIVGYPTSFMSLR
CLLSDELSNIAMQVRKLVGTQHPLLTTARGLVHDSWNSLQLRGLVVLLISKAAGPSSVNTSCQNYDMVSG
IYSCQRSLAEITELIHIALLVHRGIVNLNELQSSDGPLKDMQFGNKIAILSGDFLLANACNGLALLQNTK
VVELLASALMDLVQGVYHENSTSKESYITDDIGISTWKEQTFLSHGALLAKSCQAAMELAKHDAEVQNMA
FQYGKHMAMSHKINSDVQPFIKEKTSDSMTFNLNSAPVVLHQEFLGRDLWIKQIGEAQEKGRLDYAKLRE
RIKAGKGVTSAIDLCRYHGNKALEALESFPPSEARSALENIVFAVTRFS
(SEQ ID NO: 2) human PDSS2 Sequence:
>XP_011534258.1 PDSS2 [organism = Homo sapiens] [GeneID = 57107] [isoform = X1]
MNFRQLLLHLPRYLGASGSPRRLWWSPSLDTISSVGSWRGRSSKSPAHWNQVVSEAEKIVGYPTSFMSLR
CLLSDELSNIAMQVRKLVGTQHPLLTTARGLVHDSWNSLQLRGLVVLLISKAAGPSSVNTSCQNYDMVSG
IYSCQRSLAEITELIHIALLVHRGIVNLNELQSSDGPLKDMQFGNKIAILSGDFLLANACNGLALLQNTK
VVELLASALMDLVQGVYHENSTSKESYITDDIGISTWKEQTFLSHGALLAKSCQAAMELAKHDAEVQNMA
FQYGKHMAMSHKINSDVQPFIKEKTSDSMTFNLNSAPVVLHQEFLGRDLWIKQIGEAQEKGRLDYAKERG
LAVTQTGDAFFMTQRMPLGFLITEALDNGRDFHWKMAKRKIWCTNKSTPGPSNGSATNSMQNLLRERIKA
GKGVTSAIDLCRYHGNKALEALESFPPSEARSALENIVFAVTRFS
(SEQ ID NO: 3) human PDSS2 Sequence:
>XP_011534259.1 PDSS2 [organism = Homo sapiens] [GeneID = 57107] [isoform = X2]
MNFRQLLLHLPRYLGASGSPRRLWWSPSLDTISSVGSWRGRSSKSPAHWNQVVSEAEKIVGYPTSFMSLR
CLLSDELSNIAMQVRKLVGTQHPLLTTARGLVHDSWNSLQLRGLVVLLISKAAGPSSVNTSCQNYDMVSG
IYSCQRSLAEITELIHIALLVHRGIVNLNELQSSDGPLKDMQFGNKIAILSGDFLLANACNGLALLQNTK
VVELLASALMDLVQGVYHENSTSKESYITDDIGISTWKEQTFLSHGALLAKSCQAAMELAKHDAEVQNMA
FQYGKHMAMSHKAQEKGRLDYAKERGLAVTQTGDAFFMTQRMPLGFLITEALDNGRDFHWKMAKRKIWCT
NKSTPGPSNGSATNSMQNLLRERIKAGKGVTSAIDLCRYHGNKALEALESFPPSEARSALENIVFAVTRFS
(SEQ ID NO: 4) human PDSS2 Sequence:
>XP_011534260.1 PDSS2 [organism = Homo sapiens] [GeneID = 57107] [isoform = X3]
MNFRQLLLHLPRYLGASGSPRRLWWSPSLDTISSVGSWRGRSSKSPAHWNQVVSEAEKIVGYPTSFMSLR
CLLSDELSNIAMQVRKLVGTQHPLLTTASQRSLAEITELIHIALLVHRGIVNLNELQSSDGPLKDMQFGN
KIAILSGDFLLANACNGLALLQNTKVVELLASALMDLVQGVYHENSTSKESYITDDIGISTWKEQTFLSH
GALLAKSCQAAMELAKHDAEVQNMAFQYGKHMAMSHKINSDVQPFIKEKTSDSMTFNLNSAPVVLHQEFL
GRDLWIKQIGEAQEKGRLDYAKERGLAVTQTGDAFFMTQRMPLGFLITEALDNGRDFHWKMAKRKIWCTN
KSTPGPSNGSATNSMQNLLRERIKAGKGVTSAIDLCRYHGNKALEALESFPPSEARSALENIVFAVTRFS
(SEQ ID NO: 5) human PDSS2 Sequence:
>XP_011534261.1 PDSS2 [organism = Homo sapiens] [GeneID = 57107] [isoform = X6]
MNFRQLLLHLPRYLGASGSPRRLWWSPSLDTISSVGSWRGRSSKSPAHWNQVVSEAEKIVGYPTSFMSLR
CLLSDELSNIAMQVRKLVGTQHPLLTTARGLVHDSWNSLQLRGLVVLLISKAAGPSSVNTSCQNYDMVSG
IYSCQRSLAEITELIHIALLVHRGIVNLNELQSSDGPLKDMQFGNKIAILSGDFLLANACNGLALLQNTK
VVELLASALMDLVQGVYHENSTSKESYITDDIGISTWKEQTFLSHGALLAKSCQAAMELAKHDAEVQNMA
FQYGKHMAMSHKAQEKGRLDYAKLRERIKAGKGVTSAIDLCRYHGNKALEALESFPPSEARSALENIVFA
VTRFS
(SEQ ID NO: 6) human PDSS2 Sequence:
>XP_011534262.1 PDSS2 [organism = Homo sapiens] [GeneID = 57107] [isoform = X8]
MVSGIYSCQRSLAEITELIHIALL VHRGIVNLNELQSSDGPLKDMQFGNKIAILSGDFLLANACNGLALL
QNTKVVELLASALMDLVQGVYHENSTSKESYITDDIGISTWKEQTFLSHGALLAKSCQAAMELAKHDAEV
QNMAFQYGKHMAMSHKINSDVQPFIKEKTSDSMTFNLNSAPVVLHQEFLGRDLWIKQIGEAQEKGRLDYA
KERGLAVTQTGDAFFMTQRMPLGFLITEALDNGRDFHWKMAKRKIWCTNKSTPGPSNGSATNSMQNLLRE
RIKAGKGVTSAIDLCRYHGNKALEALESFPPSEARSALENIVFAVTRFS
(SEQ ID NO: 7) human PDSS2 Sequence:
>XP_011534263.1 PDSS2 [organism = Homo sapiens] [GeneID = 57107] [isoform = X11]
MNFRQLLLHLPRYLGASGSPRRLWWSPSLDTISSVGSWRGRSSKSPAHWNQVVSEAEKIVGYPTSFMSLR
CLLSDELSNIAMQVRKLVGTQHPLLTTARGLVHDSWNSLQLRGLVVLLISKAAGPSSVNTSCQNYDMVSG
IYSCQRSLAEITELIHIALLVHRGIVNLNELQSSDGPLKDMQFGNKIAILSGDFLLANACNGLALLQNTK
VVELLASALMDLVQGVYHENSTSKAQEKGRLDYAKLRERIKAGKGVTSAIDLCRYHGNKALEALESFPPS
EARSALENIVFAVTRFS
(SEQ ID NO: 8) human PDSS2 Sequence:
>XP_011534265.1 PDSS2 [organism = Homo sapiens] [GeneID = 57107] [isoform = X14]
MNFRQLLLHLPRYLGASGSPRRLWWSPSLDTISSVGSWRGRSSKSPAHWNQVVSEAEKIVGYPTSFMSLR
CLLSDELSNIAMQVRKLVGTQHPLLTTARGLVHDSWNSLQLRGLVVLLISKAAGPSSVNTSCQNYDMVSG
IYSCQRSLAEITELIHIALLVHRGIVNLNELQSSDGPLKDMQFGNKIAILSGDFLLANACNGLALLQNTK
SFSFNGPIAIYQMGDCESAWILSKHPRALS
(SEQ ID NO: 9) human PDSS2 Sequence:
>XP_016866571.1 PDSS2 [organism = Homo sapiens] [GeneID = 57107] [isoform = X5]
MNFRQLLLHLPRYLGASGSPRRLWWSPSLDTISSVGSWRGRSSKSPAHWNQVVSEAEKIVGYPTSFMSLR
CLLSDELSNIAMQVRKLVGTQHPLLTTARGLVHDSWNSLQLRGLVVLLISKAAGPSSVNTSCQNYDMVSG
IYSCQRSLAEITELIHIALLVHRGIVNLNELQSSDGPLKDMQFGNKIAILSGDFLLANACNGLALLQNTK
VVELLASALMDLVQGVYHENSTSKESYITDDIGISTWKEQTFLSHGALLAKSCQAAMELAKHDAEVQNMA
FQYGKHMAMSHKINSDVQPFIKEKTSDSMTFNLNSAPVVLHQEFLGRDLWIKQIGESSSTEVQLSNFQQM
LLSTLLSTA
(SEQ ID NO: 10) human PDSS2 Sequence:
>XP_047275052.1 PDSS2 [organism = Homo sapiens] [GeneID = 57107] [isoform = X4]
MNFRQLLLHLPRYLGASGSPRRLWWSPSLDTISSVGSWRGRSSKSPAHWNQVVSEAEKIVGYPTSFMSLR
CLLSDELSNIAMQVRKLVGTQHPLLTTARGLVHDSWNSLQLRGLVVLLISKAAGPSSVNTSCQNYDMVSG
IYSCQRSLAEITELIHIALLVHRGIVNLNELQSSDGPLKDMQFGNKIAILSGDFLLANACNGLALLQNTK
VVELLASALMDLVQGVYHENSTSKAQEKGRLDYAKERGLAVTQTGDAFFMTQRMPLGFLITEALDNGRDF
HWKMAKRKIWCTNKSTPGPSNGSATNSMQNLLRERIKAGKGVTSAIDLCRYHGNKALEALESFPPSEARS
ALENIVFAVTRFS
(SEQ ID NO: 11) human PDSS2 Sequence:
>XP_047275053.1 PDSS2 [organism = Homo sapiens] [GeneID = 57107] [isoform = X7]
MNFRQLLLHLPRYLGASGSPRRLWWSPSLDTISSVGSWRGRSSKSPAHWNQVVSEAEKIVGYPTSFMSLR
CLLSDELSNIAMQVRKLVGTQHPLLTTASQRSLAEITELIHIALLVHRGIVNLNELQSSDGPLKDMQFGN
KIAILSGDFLLANACNGLALLQNTKVVELLASALMDLVQGVYHENSTSKESYITDDIGISTWKEQTFLSH
GALLAKSCQAAMELAKHDAEVQNMAFQYGKHMAMSHKINSDVQPFIKEKTSDSMTFNLNSAPVVLHQEFL
GRDLWIKQIGEAQEKGRLDYAKLRERIKAGKGVTSAIDLCRYHGNKALEALESFPPSEARSALENIVFAV
TRFS
(SEQ ID NO: 12) human PDSS2 Sequence:
>XP_047275054.1 PDSS2 [organism = Homo sapiens] [GeneID = 57107] [isoform = X9]
MRDSGLVQAGWPRGLVHDSWNSLQLRGLVVLLISKAAGPSSVNTSCQNYDMVSGIYSCQRSLAEITELIH
IALLVHRGIVNLNELQSSDGPLKDMQFGNKIAILSGDFLLANACNGLALLQNTKVVELLASALMDLVQGV
YHENSTSKESYITDDIGISTWKEQTFLSHGALLAKSCQAAMELAKHDAEVQNMAFQYGKHMAMSHKINSD
VQPFIKEKTSDSMTFNLNSAPVVLHQEFLGRDLWIKQIGEAQEKGRLDYAKLRERIKAGKGVTSAIDLCR
YHGNKALEALESFPPSEARSALENIVFAVTRFS
(SEQ ID NO: 13) human PDSS2 Sequence:
>XP_047275055.1 PDSS2 [organism = Homo sapiens] [GeneID = 57107] [isoform = X10]
MNFRQLLLHLPRYLGASGSPRRLWWSPSLDTISSVGSWRGRSSKSPAHWNQVVSEAEKIVGYPTSFMSLR
CLLSDELSNIAMQVRKLVGTQHPLLTTASQRSLAEITELIHIALLVHRGIVNLNELQSSDGPLKDMQFGN
KIAILSGDFLLANACNGLALLQNTKVVELLASALMDLVQGVYHENSTSKESYITDDIGISTWKEQTFLSH
GALLAKSCQAAMELAKHDAEVQNMAFQYGKHMAMSHKAQEKGRLDYAKLRERIKAGKGVTSAIDLCRYHG
NKALEALESFPPSEARSALENIVFAVTRFS
(SEQ ID NO: 14) human PDSS2 Sequence:
>XP_047275056.1 PDSS2 [organism = Homo sapiens] [GeneID = 57107] [isoform = X12]
MQFGNKIAILSGDFLLANACNGLALLQNTKVVELLASALMDLVQGVYHENSTSKESYITDDIGISTWKEQ
TFLSHGALLAKSCQAAMELAKHDAEVQNMAFQYGKHMAMSHKINSDVQPFIKEKTSDSMTFNLNSAPVVL
HQEFLGRDLWIKQIGEAQEKGRLDYAKERGLAVTQTGDAFFMTQRMPLGFLITEALDNGRDFHWKMAKRK
IWCTNKSTPGPSNGSATNSMQNLLRERIKAGKGVTSAIDLCRYHGNKALEALESFPPSEARSALENIVFA
VTRFS
(SEQ ID NO: 15) human PDSS2 Sequence:
>XP_047275057.1 PDSS2 [organism = Homo sapiens] [GeneID = 57107] [isoform = X13]
MNFRQLLLHLPRYLGASGSPRRLWWSPSLDTISSVGSWRGRSSKSPAHWNQVVSEAEKIVGYPTSFMSLR
CLLSDELSNIAMQVRKLVGTQHPLLTTASQRSLAEITELIHIALLVHRGIVNLNELQSSDGPLKDMQFGN
KIAILSGDFLLANACNGLALLQNTKVVELLASALMDLVQGVYHENSTSKAQEKGRLDYAKLRERIKAGKG
VTSAIDLCRYHGNKALEALESFPPSEARSALENIVFAVTRES
(SEQ ID NO: 16) Mouse Nucleotide Sequence (1206 nt):
ATGAGCCTCCGGCAGCTGCTGTTGCGCTTGTCCGGTTACCTCGGGGCTTCA
GGTCCCCCCAGTCGCCACTGGTGGTACTTCAGATCCCTCGACAGCATCTCC
TCGGCGGGCTCCTGGCGCGGGCGCTCCTCCAGGTCACCGGCCCATTGGAA
CCAGGTGGTGTCCGAGGCGGAGAAGATCGTGGGCTACCCCGCATCCTTCA
TGAGCCTGCGCTGCCTGCTGAGCGACGAGCTCAGCAATATCGCCATGCAG
GTGCGGAAGCTGGTGGGGACGGGACACCCTCTGCTTACCACTGCCAGGGC
CCTCGTGCACGACAGCCGGCATAACCTACAACTGCGGGGCCTGGTCGTGC
TCCTCATATCAAAGGCTGCGGGGCCCAGCACTCGGAACGCTGCGTGTCAG
AACTACGACATGGTCAGTGGGGTATACTCATGTCAAAGAAGTTTGGCAGA
GATCACAGAACTTATCCATACTGCTCTCCTGGTGCATCGTGGGATAGTAAA
CTTAAGTGAATTACAGTCATCTGATGGACCACTGAAAGACATGCAGTTTG
GAAACAAAATAGCTATCCTGAGTGGAGACTTCCTTCTAGCAAATGCATGC
AATGGACTAGCTCTTCTACAGAACACCAAGGTTGTGGAGCTTTTATCAAGT
GCTCTTATGGACTTGGTGCATGGAGTATACCAGGAGAACTCTGCTTCCACC
AAGGAAAATTCTATCCCAGATGATATTGGAATCTCGACCTGGAAGGAGCA
GACTTTCCTGTCCCATTGTGCCTTGCTAGCGAAGAGCTGCCAGGCTGCAAT
GGAGTTAGCAAAGCATGATGCTGCGGTCCAAGACATGGCATTCCAGTATG
GGAAGCACATGGCCATGAGTCACAAGATCAATGCTGACCTCCAGCCTTTT
ATTAAAGACAAGGCCAGTGACTCTAAGACTTTTAACCTAAACTCAGCACC
TGTAGTCTTACATCAGGAGTTTCTTGGAAGAGATTTGTGGATTAAGCAGAT
TGGAGAGGCTCAAGAGAAAGGAAGCTTGAACTACAGTAAGTTGCGAGAA
ACAATCAAAGCTGGCAAAGGTGTGACTTCAGCTATTGACCTGTGTCGTTA
CCATGGAAACAAGGCACTAGAGGCCCTGGAGAGCTTCCCTCCCTCAGAGG
CCAGATCGGCTTTAGAAAACATTGTGTTTGCTGTGACCAGATTTTCTTGA
(SEQ ID NO: 17) Mouse Translation (401 aa):
MSLRQLLLRLSGYLGASGPPSRHWWYFRSLDSISSAGSWRGRSSRSPAHWNQ
VVSEAEKIVGYPASFMSLRCLLSDELSNIAMQVRKLVGTGHPLLTTARALVH
DSRHNLQLRGLVVLLISKAAGPSTRNAACQNYDMVSGVYSCQRSLAEITELIH
TALLVHRGIVNLSELQSSDGPLKDMQFGNKIAILSGDFLLANACNGLALLQNT
KVVELLSSALMDLVHGVYQENSASTKENSIPDDIGISTWKEQTFLSHCALLAK
SCQAAMELAKHDAAVQDMAFQYGKHMAMSHKINADLQPFIKDKASDSKTF
NLNSAPVVLHQEFLGRDLWIKQIGEAQEKGSLNYSKLRETIKAGKGVTSAIDL
CRYHGNKALEALESFPPSEARSALENIVFAVTRFS
(SEQ ID NO: 18) Mouse PDSS2 RNA:
ATGCCTCCAACATGGCGCCCCCCATGTAGGCACTGGCTGTGGTTACCAGC
GCAGGTATCCAGTGCAGTTCTGAGCAGCCGCTGGGGTTACGCCAGTCTGC
GCTGCAGAAGCAGCAAACGTTATTCCTGGTTACGGCTCCAGAAAGCCATA
CTTCTCTGAGGGTTTGGAATCGCCGTCTCCTGTTAGATTCTGAAAGTTTCT
CCTCAGCCCTTGTTTGCTTCCCGTGTCATTTCTGGCCACCTTCCTCAGGTCC
CCGACCCTCTCAGACTCAAAACTATGAGCCTCCGGCAGCTGCTGTTGCGCT
TGTCCGGTTACCTCGGGGCTTCAGGTCCCCCCAGTCGCCACTGGTGGTACT
TCAGATCCCTCGACAGCATCTCCTCGGCGGGCTCCTGGCGCGGGCGCTCCT
CCAGGTCACCGGCCCATTGGAACCAGGTGGTGTCCGAGGCGGAGAAGATC
GTGGGCTACCCCGCATCCTTCATGAGCCTGCGCTGCCTGCTGAGCGACGA
GCTCAGCAATATCGCCATGCAGGTGCGGAAGCTGGTGGGGACGGGACACC
CTCTGCTTACCACTGCCAGGGCCCTCGTGCACGACAGCCGGCATAACCTA
CAACTGCGGGGCCTGGTCGTGCTCCTCATATCAAAGGCTGCGGGGCCCAG
CACTCGGAACGCTGCGTGTCAGAACTACGACATGGTCAGTGGGGTATACT
CATGTCAAAGAAGTTTGGCAGAGATCACAGAACTTATCCATACTGCTCTC
CTGGTGCATCGTGGGATAGTAAACTTAAGTGAATTACAGTCATCTGATGG
ACCACTGAAAGACATGCAGTTTGGAAACAAAATAGCTATCCTGAGTGGAG
ACTTCCTTCTAGCAAATGCATGCAATGGACTAGCTCTTCTACAGAACACCA
AGGTTGTGGAGCTTTTATCAAGTGCTCTTATGGACTTGGTGCATGGAGTAT
ACCAGGAGAACTCTGCTTCCACCAAGGAAAATTCTATCCCAGATGATATT
GGAATCTCGACCTGGAAGGAGCAGACTTTCCTGTCCCATTGTGCCTTGCTA
GCGAAGAGCTGCCAGGCTGCAATGGAGTTAGCAAAGCATGATGCTGCGGT
CCAAGACATGGCATTCCAGTATGGGAAGCACATGGCCATGAGTCACAAGA
TCAATGCTGACCTCCAGCCTTTTATTAAAGACAAGGCCAGTGACTCTAAG
ACTTTTAACCTAAACTCAGCACCTGTAGTCTTACATCAGGAGTTTCTTGGA
AGAGATTTGTGGATTAAGCAGATTGGAGAGGCTCAAGAGAAAGGAAGCT
TGAACTACAGTAAGTTGCGAGAAACAATCAAAGCTGGCAAAGGTGTGACT
TCAGCTATTGACCTGTGTCGTTACCATGGAAACAAGGCACTAGAGGCCCT
GGAGAGCTTCCCTCCCTCAGAGGCCAGATCGGCTTTAGAAAACATTGTGT
TTGCTGTGACCAGATTTTCTTGACACCAAATTAAAAAGACACTATTGTTCC
TTAGCTGAAAAACCCAGAGAATGAGGCGGACAGGAGAGCTTTTGGTGATG
AAATAAGTAACGTGTTTAATGACTTTAGAAATATATGTATTTTTTTTTTCTG
TCCTTTATCTGACTGCTTAATGAATCTATCCTTTGGAACCACTACAAACAA
AACGAGAAGGAAAAAGGTCACGCGGTTTTCACTTGTCACGCCAGAGCACA
CTTGAGGCTTCAGCAACAGAAATAATTAATGAGACTCGCTCCCGTGACCT
CAGCAAATGG

### Formulations and Pharmaceutical Compositions

In alternative embodiments, provided are pharmaceutical formulations or compositions comprising CD8 T cells which, upon administration in vivo (to an individual in need thereof) result in the individuals having improved metabolic fitness and increased accumulation in vivo in response to infection and tumors, metabolically enhanced chimeric antigen receptor (CAR)-T cell function and persistence in vitro or in vivo, and/or enhanced CAR-T cell efficacy in cancer immunotherapy.

The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, for example, the latest edition of Remington's Pharmaceutical Sciences, Maack Publishing Co., Easton PA (“Remington's”).

For example, in alternative embodiments, these compositions used to practice methods and uses as provided herein are formulated in a buffer, in a saline solution, in a powder, an emulsion, in a vesicle, in a liposome, in a nanoparticle, in a nanolipoparticle and the like. In alternative embodiments, the compositions can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature.

### Human PDSS2 Enzyme Sequences

In alternative embodiments, heterologous nucleic acids encoding a PDSS2 enzyme inserted into and expressed in T cells and CAR-T cells, wherein the PDSS2 enzyme encoded by the nucleic acid has a sequence as set forth in any of SEQ ID NO: 1 to SEQ ID NO:15, or the heterologous nucleic acid encodes a protein having an amino acid sequence as set forth in any of SEQ ID NO: 1 to SEQ ID NO:15, or the nucleic acid has between about 90% to 99% sequence identity to any of SEQ ID NO:1 to SEQ ID NO:15, and encoding a polypeptide having PDSS2 activity; or a polypeptide having about 92%, 95% or 97% sequence identity to a sequence as set forth in any of SEQ ID NO: 1 to SEQ ID NO:15, and having PDSS2 activity, wherein optionally the sequence identities (SEQ IDs) are determined by analysis with a sequence comparison algorithm or by a visual inspection, and optionally the sequence comparison algorithm is a BLAST VERSION 2.2.2™ algorithm where a filtering setting is set to blastall -p blastp -d “nr pataa” -F F, and all other options are set to default.

Various sequence comparison programs identified in this patent specification are particularly contemplated for use in this embodiment. Protein and/or nucleic acid sequence homologies may be evaluated using any of the variety of sequence comparison algorithms and programs known in the art. Such algorithms and programs include, but are by no means limited to, TBLASTN™, BLASTP™, FASTA™, TFASTA™ and CLUSTALW™ (see, e.g., Pearson and Lipman, Proc. Natl. Acad. Sci. USA 85(8):2444-2448, 1988; Altschul et al., J. Mol. Biol. 215(3):403-410, 1990; Thompson Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993).

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequence for comparison are well-known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol 48:443, 1970, by the search for similarity method of person & Lipman, Proc. Nat'l. Acad. Sci. USA 85:2444, 1988, by computerized implementations of these algorithms (GAP™, BESTFIT™, FASTA™ and TFASTA™ in the Wisconsin Genetics Software Package, Genetics Computer Group, 575 Science Dr., Madison, WI), or by manual alignment and visual inspection. Other algorithms for determining homology or identity include, for example, in addition to a BLAST™ program (Basic Local Alignment Search Tool at the National Center for Biological Information), ALIGN™, AMAS™ (Analysis of Multiply Aligned Sequences), AMPS™ (Protein Multiple Sequence Alignment), ASSET (Aligned Segment Statistical Evaluation Tool), BANDS™, BESTSCOR™, BIOSCAN™ (Biological Sequence Comparative Analysis Node), BLIMPS™ (BLocks IMProved Searcher), FASTA™, Intervals & Points, BMB™, CLUSTAL V™, CLUSTAL W™, CONSENSUS™, LCONSENSUS™, WCONSENSUS™, Smith-Waterman algorithm, DARWIN™, Las Vegas algorithm, FNAT™ (Forced Nucleotide Alignment Tool), FRAMEALIGN™, FRAMESEARCH™, DYNAMIC™, FILTER™, FSAP™ (Fristensky Sequence Analysis Package), GAP™ (Global Alignment Program), GENAL™, GIBBS™, GENQUEST™, ISSC™ (Sensitive Sequence Comparison), LALIGN™ (Local Sequence Alignment), LCP™ (Local Content Program), MACAW™ (Multiple Alignment Construction & Analysis Workbench), MAP™ (Multiple Alignment Program), MBLKP™, MBLKN™, PIMA™ (Pattern-Induced Multi-sequence Alignment), SAGA™ (Sequence Alignment by Genetic Algorithm) and WHAT-IF™ Such alignment programs can also be used to screen genome databases to identify polynucleotide sequences having substantially identical sequences. A number of genome databases are available, for example, a substantial portion of the human genome is available as part of the Human Genome Sequencing Project (Gibbs, 1995). At least twenty-one other genomes have already been sequenced, including, for example, M. genitalium (Fraser et al., 1995), M. Jannaschii (Bult et al., 1996), H. influenzae (Fleischmann et al., 1995), E. coli (Blattner et al., 1997) and yeast (S. cerevisiae) (Mewes et al., 1997) and D. melanogaster (Adams et al., 2000). Significant progress has also been made in sequencing the genomes of model organism, such as mouse, C. elegans and Arabadopsis sp. Several databases containing genomic information annotated with some functional information are maintained by different organizations and may be accessible via the internet.

One example of a useful algorithm is BLAST™ and BLAST 2.0™ algorithms, which are described in Altschul et al., Nuc. Acids Res. 25:3389-3402, 1977 and Altschul et al., J. Mol. Biol. 215:403-410, 1990, respectively. Software for performing BLAST™ analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST™ algorithm parameters W, T and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3 and expectations (E) of 10 and the BLOSUM62 scoring matrix (see Henikoff & Henikoff, Proc. Natl. Acad. Sci. USA 89:10915, 1989) alignments (B) of 50, expectation (E) of 10, M=5, N=−4 and a comparison of both strands.

The BLAST™ algorithm also performs a statistical analysis of the similarity between two sequences (see, e.g., Karlin & Altschul, Proc. Natl. Acad. Sci. USA 90:5873, 1993). One measure of similarity provided by BLAST™ algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more in one aspect less than about 0.01 and most in one aspect less than about 0.001.

In one aspect, protein and nucleic acid sequence homologies are evaluated using the Basic Local Alignment Search Tool (“BLAST™”) In particular, five specific BLAST™ programs are used to perform the following task:
(1) BLASTP™ and BLAST3™ compare an amino acid query sequence against a protein sequence database;
(2) BLASTN™ compares a nucleotide query sequence against a nucleotide sequence database;
(3) BLASTX™ compares the six-frame conceptual translation products of a query nucleotide sequence (both strands) against a protein sequence database;
(4) TBLASTN™ compares a query protein sequence against a nucleotide sequence database translated in all six reading frames (both strands); and
(5) TBLASTX™ compares the six-frame translations of a nucleotide query sequence against the six-frame translations of a nucleotide sequence database.

The BLAST™ programs identify homologous sequences by identifying similar segments, which are referred to herein as “high-scoring segment pairs,” between a query amino or nucleic acid sequence and a test sequence which is in one aspect obtained from a protein or nucleic acid sequence database. High-scoring segment pairs are in one aspect identified (i.e., aligned) by means of a scoring matrix, many of which are known in the art. In one aspect, the scoring matrix used is the BLOSUM62™ matrix (Gonnet (1992) Science 256:1443-1445; Henikoff and Henikoff (1993) Proteins 17:49-61). Less in one aspect, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation). BLAST™ programs are accessible through the U.S. National Library of Medicine.

The parameters used with the above algorithms may be adapted depending on the sequence length and degree of homology studied. In some aspects, the parameters may be the default parameters used by the algorithms in the absence of instructions from the user.

### In Vivo Delivery of PDSS2-Encoding Nucleic Acids

In alternative embodiments, provided are compositions and methods for delivering to a T cell or a CAR-T cell a decaprenyl-diphosphate synthase subunit 2 (PDSS2) enzyme-encoding nucleic acid, or vectors or recombinant viruses having contained therein these nucleic acids. In alternative embodiments, the nucleic acids, vectors or recombinant viruses are designed for in vivo or CNS delivery and expression.

In alternative embodiments, provided are compositions and methods for the delivery and controlled expression of a PDSS2-encoding nucleic acid or gene, or an expression vehicle (for example, vector, recombinant virus, and the like) comprising (having contained therein) a PDSS2-encoding nucleic acid or gene, that results in a PDSS2 protein being synthesized in an T cell or CAR-T cell.

In alternative embodiments, the provided are methods for being able to turn on and turn off a PDSS2-encoding nucleic acids or gene expression easily and efficiently for tailored treatments and insurance of optimal safety.

In alternative embodiments, a PDSS2 protein or proteins expressed by the a PDSS2-encoding nucleic acids or gene(s) have a beneficial or favorable effects (for example, therapeutic or prophylactic) on a tissue or an organ, for example, an anti-cancer effect.

In alternative embodiments, provided are expression vehicles, vectors, recombinant viruses and the like for in vivo expression of an a PDSS2-encoding nucleic acids or gene to practice the methods as provide herein.

Alternative embodiments comprise use of “expression cassettes” comprising or having contained therein a nucleotide sequence used to practice methods provided herein, for example, an a PDSS2-encoding nucleic acid, which can be capable of affecting expression of the nucleic acid, for example, as a structural gene or a transcript (for example, encoding an a PDSS2 protein) in a T cell. Expression cassettes can include at least a promoter operably linked with the polypeptide coding sequence or inhibitory sequence; and, in one aspect, with other sequences, for example, transcription termination signals. Additional factors necessary or helpful in effecting expression may also be used, for example, enhancers.

In alternative aspects, expression cassettes also include plasmids, expression vectors, recombinant viruses, any form of recombinant “naked DNA” vector, and the like. In alternative aspects, a “vector” can comprise a nucleic acid that can infect, transfect, transiently or permanently transduce a cell. In alternative aspects, a vector can be a naked nucleic acid, or a nucleic acid complexed with protein or lipid. In alternative aspects, vectors can comprise viral or bacterial nucleic acids and/or proteins, and/or membranes (for example, a cell membrane, a viral lipid envelope, etc.). In alternative aspects, vectors can include, but are not limited to replicons (for example, RNA replicons, bacteriophages) to which fragments of DNA may be attached and become replicated. Vectors thus include, but are not limited to RNA, autonomous self-replicating circular or linear DNA or RNA (for example, plasmids, viruses, and the like, see, for example, U.S. Pat. No. 5,217,879), and can include both the expression and non-expression plasmids. In alternative aspects, a vector can be stably replicated by the cells during mitosis as an autonomous structure, or can be incorporated within the host's genome.

In alternative aspects, “promoters” include all sequences capable of driving transcription of a coding sequence in a cell, for example, a mammalian cell such as a retinal cell. Promoters used in the constructs provided herein include cis-acting transcriptional control elements and regulatory sequences that are involved in regulating or modulating the timing and/or rate of transcription of a nucleic acid, for example, an a PDSS2-encoding nucleic acid. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription.

In alternative embodiments, “constitutive” promoters can be those that drive expression continuously under most environmental conditions and states of development or cell differentiation. In alternative embodiments, “inducible” or “regulatable” promoters can direct expression of a nucleic acid, for example, an AIBP-encoding nucleic acid, under the influence of environmental conditions, administered chemical agents, or developmental conditions.

### Gene Therapy and Gene Delivery Vehicles

In alternative embodiments, methods of the invention comprise use of a PDSS2-encoding nucleic acid, or a PDSS2-encoding nucleic acid, transcript or message, to a T cell or T cells.

In alternative embodiments, a PDSS2-encoding nucleic acid, transcript or message is contained in a expression vehicle, vector, recombinant virus, or equivalents used to practice methods provided herein are or comprise: an adeno-associated virus (AAV), a lentiviral vector or an adenovirus vector; an AAV serotype AAV5, AAV6, AAV8 or AAV9; a rhesus-derived AAV, or the rhesus-derived AAV AAVrh. 10hCLN2; an organ-tropic AAV, or a neurotropic AAV; and/or an AAV capsid mutant or AAV hybrid serotype.

In alternative embodiments, the rhesus-derived AAV AAVrh. 10hCLN2 or equivalents thereof can be used, wherein the rhesus-derived AAV may not be inhibited by any pre-existing immunity in a human; see for example, Sondhi, et al., Hum Gene Ther. Methods. 2012 October; 23(5):324-35, Epub 2012 Nov. 6; Sondhi, et al., Hum Gene Ther. Methods. 2012 Oct. 17; teaching that direct administration of AAVrh. 10hCLN2 to the CNS of rats and non-human primates at doses scalable to humans has an acceptable safety profile and mediates significant payload expression in the CNS.

### Dosaging

The pharmaceutical compositions and formulations used to practice methods and uses as provided herein can be administered for prophylactic and/or therapeutic treatments, for example, to treat, ameliorate a cancer or tumor, and in particular, for the enhancement of CAR-T cell efficacy in cancer immunotherapy. In therapeutic applications, compositions are administered to a subject already suffering from a disease, condition, infection or defect in an amount sufficient to cure, alleviate or partially arrest the clinical manifestations of the disease, condition, infection or disease and its complications (a “therapeutically effective amount”), including for example, cancer. For example, in alternative embodiments, T cells or CAR-T cells having enhanced or increase amounts of PDSS2-expressing nucleic acid as provided herein are administered to an individual in need thereof in an amount sufficient to treat, ameliorate, protect against, reverse or decrease the severity of a cancer; or, for for metabolically enhancing CD8 T cells resulting in their having improved metabolic fitness and increased accumulation in vivo in response to infection and tumors.

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

In alternative embodiments, CD8 T cells or CAR-T cells are administered to an individual in need therein, and in alternative embodiment the dosage administered to a human comprises: a dose of about 2×10¹² cells per kg body weight (vg/kg), or between about 10¹⁰ and 10¹⁴ cells per kg body weight (vg/kg), or about 10⁹, 10¹⁰, 10¹¹, 10¹², 10¹³, 10¹⁴, 10¹⁵, or more cells, which can be administered as a single dosage or in multiple dosages, as needed. In alternative embodiments, these dosages are administered intravitreally, IM, IV, or intrathecally. In alternative embodiments, the vectors are delivered as formulations or pharmaceutical preparations, for example, where the cells are contained in a nanoparticle, a particle, a micelle or a liposome or lipoplex, a polymersome, a polyplex or a dendrimer.

In alternative embodiments, these dosages are administered once a day, once a week, or any variation thereof as needed to maintain in vivo levels of engineered CD8 T cells as provided herein, which can be monitored by measuring levels of engineered CD8 T cells or by monitoring of therapeutic effect, for example, to treat, ameliorate, protect against, reverse or decrease the severity of a cancer or to improve an immune response, wherein optionally the immune response is an anti-cancer immune response. The dosage regimen also takes into consideration pharmacokinetics parameters well known in the art, for example, cell clearance (see, for example, Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra).

Single or multiple administrations of formulations can be given depending on the dosage and frequency as required and tolerated by the patient. The formulations should provide a sufficient quantity of engineered CD8 T cells as provided herein to effectively treat, prevent or ameliorate a conditions, diseases or symptoms as described herein.

The methods as provided herein can further comprise co-administration with other drugs or pharmaceuticals, for example, compositions for treating any neurological or neuromuscular disease, condition, infection or injury, including related inflammatory and autoimmune diseases and conditions, and the like. For example, the methods and/or compositions and formulations as provided herein can be co-formulated with and/or co-administered with, fluids, antibiotics, cytokines, immunoregulatory agents, anti-inflammatory agents, pain alleviating compounds, complement activating agents, such as peptides or proteins comprising collagen-like domains or fibrinogen-like domains (for example, a ficolin), carbohydrate-binding domains, and the like and combinations thereof.

### CRISPR Gene Therapy

In alternative embodiments, T cells genetically manipulated or engineered to have increased PDSS2 polypeptide expression, wherein optionally the T cell is a chimeric antigen receptor (CAR)-T cell, and optionally the CAR-T cell targets cancer, wherein the T cells can be delivered in vivo or ex vivo, or PDSS2 polypeptide-expressing nucleic acids or expression vehicles containing PDSS2 polypeptide-expressing nucleic acids, which can be delivered to T cells in vivo or ex vivo, are used to practice embodiments as provided herein, and in alternative embodiments, these T cells are manipulated to express or overexpress PDSS2 polypeptides using a CRISPR system such as a CRISPR-Cas9 system. For example, in one embodiment, a CRISPR system increases expression of a homologous PDSS2 gene, message and/or polypeptide, and in another embodiment a CRISPR system is used to implant a heterologous PDSS2-expressing nucleic acid.

Delivery of PDSS2 exogenous nucleic acids, Cas9, sgRNA, and associated complexes into cells such as T cells can occur using viral and non-viral systems: for example, electroporation of DNA, RNA, or ribonucleocomplexes; chemical transfection techniques utilizing lipids and peptides (particularly to introduce sgRNAs in complex with Cas9 into cells); nanoparticle-based delivery for transfection, and the like. Some categories of cells are more difficult to transfect, including stem cells, neurons, and hematopoietic cells, and these require more efficient delivery systems, such as those based on lentivirus (LVs), adenovirus (AdV), and adeno-associated virus (AAV).

Variants of CRISPR-Cas9 an be used to allow gene activation or genome editing with an external trigger such as light or small molecules: including photoactivatable CRISPR systems developed by fusing light-responsive protein partners with an activator domain and a dCas9 for gene activation, or by fusing similar light-responsive domains with two constructs of split-Cas9, or by incorporating caged unnatural amino acids into Cas9, or by modifying the guide RNAs with photocleavable complements for genome editing.

In alternative embodiments, “dead” versions of Cas9 (dCas9) are used to eliminate CRISPR's DNA-cutting ability while preserving its ability to target desirable sequences. Various regulatory factors can be added to dCas9s, enabling turning any gene on or off or adjust its level of activity. Like RNAi, CRISPR interference (CRISPRi) can turn off genes in a reversible fashion by targeting, but not cutting a site. The targeted site is methylated, epigenetically modifying the gene. This modification inhibits transcription. These precisely placed modifications may then be used to regulate the effects on gene expression (for example, XCL1 exogenous nucleic acids) and DNA dynamics after the inhibition of certain genome sequences within DNA.

In alternative embodiments, CRISPR-Cas13 fused to deaminases is used to direct mRNA editing; for example, Cas7-11, is better suited for therapeutic RNA editing than Cas13, and enables sufficiently targeted cuts.

In alternative embodiments, any CRISPR system can be used to practice methods as provided herein, for example, as described in US 2022 0387560 A1, which describes methods of treating and/or correcting ocular disease in vivo using an Adeno-associated virus (AAV) system, where the AAV system employs a nucleic acid encoding a CRISPR-Cas9 system for targeted gene disruption or correction; or US 2022 0389398 A1 that describes using engineered CRISPR/Cas effector enzymes, such as Cas13 (Cas13d, Cas13e, or Cas13f) that maintain guide-sequence-specific endonuclease activity and lack guide-sequence-independent collateral endonuclease activity; or US 2023 0029506 A1, which describes therapeutic applications of the crispr-cas systems and compositions for genome editing; or, US 2020 0340012 A1, which describes a modular CRISPR-Cas9 architecture that allows better delivery, specificity and selectivity of gene editing; or U.S. Pat. No. 8,771,945, which describes CRISPR-Cas systems and methods for altering expression of gene products; or WO 2023 283420 A2 which describes therapeutic gene silencing with crispr-cas13.

In alternative embodiments, design of a nucleic acid encoding PDSS2 exogenous nucleic acids, or for increasing homologous PDSS2 expression, can be based on the nucleic acid sequence of the gene or transcript of human PDSS2 (see for example genes encoding any of SEQ ID NO: 1 to SEQ ID NO:15).

### Products of Manufacture and Kits

Provided are products of manufacture and kits for practicing methods as provided herein, which can optionally comprise recombinantly or genetically engineered cells such as CD 8 cells and/or CAR-T cells treated in vitro/ex vivo to have increased PDSS2 message. In alternative embodiments, products of manufacture and kits as provided herein further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

A number of embodiments of the invention have been described. Nevertheless, it can be understood that various modifications may be made without departing from the spirit and scope of the invention. Accordingly, other embodiments are within the scope of the following claims.

1: A CD8 cell or a CAR-T cell or a pharmaceutical formulation comprising CD8 cell or a CAR-T cell, wherein the CD8 cell or a CAR-T cell is genetically modified to have increased expression of, or substantially increased expression or activity of, or more than wild type levels of expression or activity of, a decaprenyl-diphosphate synthase subunit 2 (PDSS2) enzyme or gene.

2: The genetically modified CD8 cell or CAR-T cell of claim 1, wherein the CD8 cell or CAR-T cell is formulated as a pharmaceutical, or formulated for in vivo administration.

3: A CD8 cell or CAR-T cell or a pharmaceutical formulation comprising a CAR-T cell, wherein the CAR-T cell comprises or has contained therein a compound or composition capable of generating a phenotype of: increased expression of, or substantially increased expression or activity of, or more than wild type levels of expression or activity of, a decaprenyl-diphosphate synthase subunit 2 (PDSS2) enzyme or gene.

4: The CD8 cell or CAR-T cell of claim 1, wherein the CD8 cell or CAR-T cell further comprises, or has implanted, a heterologous nucleic acid encoding a protein having decaprenyl-diphosphate synthase subunit 2 (PDSS2) enzyme function.

5: The CD8 cell or CAR-T cell of claim 1, wherein the PDSS2 enzyme or gene is a human enzyme or gene.

6: The CD8 cell or CAR-T cell of claim 5, wherein the PDSS2 enzyme has a sequence as set forth in any of SEQ ID NO:1 to SEQ ID NO: 15, or the heterologous nucleic acid encodes a protein having an amino acid sequence as set forth in any of SEQ ID NO: 1 to SEQ ID NO: 15.

7: The CD8 cell or CAR-T cell of claim 1, further comprising an expression vehicle or a vector having the heterologous nucleic acid contained therein, and the heterologous nucleic acid expresses a PDSS2 enzyme in the CD8 cell or CAR-T cell.

8: The CD8 cell or CAR-T cell of claim 7, wherein the expression vehicle or the vector is or comprises an expression construct, a plasmid, an expression vehicle, or a virus.

9: The CD8 cell or CAR-T cell of claim 8, wherein the expression vehicle, vector or virus is or is derived from: a herpes simplex virus, a human immunodeficiency virus (HIV), a synthetic vector, an adeno-associated virus (AAV), a lentivirus, an adenovirus or a plasmid,
and optionally the AAV is or is derived from: an AAV1, AAV2, AAV3, AAV4, AAV5, AAV6, AAV7, AAV8, AAV9, AAV10 AAV11, AAV12, pseudotyped AAV, a rhesus-derived AAV, AAVrh8, AAVrh10 and AAV-DJan AAV capsid mutant, an AAV hybrid serotype, an organ-tropic AAV, a cardiotropic AAV, or a cardiotropic AAVM41 mutant.

10: The CD8 cell or CAR-T cell of claim 1, wherein the PDSS2 enzyme-expressing nucleic acid is an RNA or a DNA molecule, or a synthetic nucleic acid.

11: The CD8 cell or CAR-T cell of claim 1, wherein the CAR-T cell is formulated or manufactured as or with a pharmaceutical, an implant, a device or is formulated for in vivo administration.

12: A product of manufacture or kit, or a pharmaceutical formulation, comprising a CD8 cell or CAR-T cell of claim 1.

13: A method for metabolically enhancing a CD8 T cell or a CAR-T cell resulting in the CD8 T cell or the CAR-T having improved metabolic fitness, and metabolically enhancing chimeric antigen receptor (CAR)-T cell function and persistence in vitro or in vivo, the method comprising inserting in the CD8 T cell or the CAR-T cell or transfecting the CD8 T cell or the CAR-T cell a heterologous nucleic acid encoding a protein having decaprenyl-diphosphate synthase subunit 2 (PDSS2) enzyme function.

14: The method of claim 13, wherein the PDSS2 enzyme has a sequence as set forth in any of SEQ ID NO:1 to SEQ ID NO:15, or the heterologous nucleic acid encodes a protein having an amino acid sequence as set forth in any of SEQ ID NO: 1 to SEQ ID NO: 15.

15: The method of claim 13, wherein the heterologous nucleic acid encoding the protein having PDSS2 enzyme function is contained in an expression vehicle or a vector.

16: The method of claim 15, wherein the expression vehicle, vector or virus is or is derived from: a herpes simplex virus, a human immunodeficiency virus (HIV), a synthetic vector, an adeno-associated virus (AAV), a lentivirus, an adenovirus or a plasmid.

17: A method for:
for metabolically enhancing CD8 T cells resulting in their having improved metabolic fitness and increased accumulation in vivo in response to infection and tumors,
metabolically enhancing chimeric antigen receptor (CAR)-T cell function and persistence in vitro or in vivo, or
enhancing CAR-T cell efficacy in cancer immunotherapy,
the method comprising:
(a) administering to an individual in need thereof a CD8 cell or a CAR-T cell having increased or enhanced expression of, or substantially more expression or activity of, or more than wild type levels of expression or activity of, a PDSS2 enzyme or gene;
(b) administering to an individual in need thereof a CD8 cell or CAR-T cell that has been engineered to have increased or enhanced expression of, or substantially more expression or activity of, or more than wild type levels of expression or activity of, a PDSS2 enzyme or gene; or
(c) administering to an individual in need thereof a CD8 cell and/or a CAR-T cell that has been genetically modified to have a phenotype comprising: having increased or enhanced expression of, or substantially more expression or activity of, or more than wild type levels of expression or activity of, a PDSS2 enzyme or gene.

18: The method of claim 17, wherein the CD8 cell or CAR-T cell is administered by intravenous injection, subcutaneous injection, intrathecal injection, or intramuscular injection.

19: The method of claim 17, wherein between about 10² and 10¹⁰ CD8 cells or CAR-T cells are administered to the individual in need thereof.

20-21. (canceled)

22: The method of claim 17, wherein in step (a):
(i) the CD8 cell or CAR-T cell has increased expression of nucleic acids encoding PDSS2, or
(ii) the CAR-T cell is contacted with nucleic acids encoding PDSS2 before in vivo administration to an individual in need thereof, during or with in vivo administration of the CD8 cell or CAR-T cell, and/or after in vivo administration of the CD8 cell or CAR-T cell.

23: The method of claim 17, wherein in step (b) the CD8 cell or CAR-T cell has increased expression of nucleic acids encoding PDSS2.
